(19) United States
(12) Patent Application Publication
(10) Pub. No.: US 2003/0215842 A1
(43) Pub. Date: Nov. 20, 2003

(54) Method for the analysis of cytosine methylation patterns

(75) Inventors: Andrew Z. Sledziewski, Shoreline, WA (US); Richard Gary Schweikhardt, Seattle, WA (US)

Correspondence Address: Davis Wright Tremaine LLP, 2600 Century Square, 1501 Fourth Avenue, Seattle, WA, 98101-1688, US

(73) Assignee: Epigenomics AG

(21) Appl. No.: 10/356,792

(22) Filed: Jan. 30, 2003

Related U.S. Application Data
(60) Provisional application No. 60/352,944, filed on Jan. 30, 2002.

(51) Int. Cl. C12Q 1/68

(52) U.S. Cl. 435/6

(57) ABSTRACT

The present invention provides a novel method for the systematic identification of differentially methylated CpG dinucleotides positions within genomic DNA sequences for use as reliable diagnostic, prognostic and/or staging markers. Particular embodiments comprise genome-wide identification of differentially methylated CpG dinucleotide sequences, further identification of neighboring differentially methylated CpG dinucleotide sequences, and confirmation of the diagnostic utility of selected differentially methylated CpG dinucleotide among a larger set of diseased and normal biological samples. The method, and kits for implementation thereof, are useful in applied assays for the diagnosis, prognosis and/or staging of conditions characterized by differential methylation.

### FIELD OF THE INVENTION

The present invention relates to genomic DNA sequences that exhibit altered CpG methylation patterns in disease states relative to normal. Particular embodiments provide a systematic method for the efficient identification, assessment and validation of differentially methylated genomic CpG dinucleotide sequences as diagnostic and/or prognostic markers.

### BACKGROUND

Significant developments in medical science have arisen over the past decade, reflecting an increased understanding of the human genome. However, even with completion of the sequencing of the Human Genome, fundamental questions remain concerning the mechanisms by which the genome is controlled and the relationship between such mechanisms and disease.

Genetic approaches. The vast majority of efforts to identify genomic abnormalities has been, and continues to be based on nucleotide sequence analysis; that is genetic based. During initial phases of the human genome project, genomic markers were linked to disease conditions by mapping. Such mapping techniques involved correlation of the incidence of a disease condition with inheritance of genomic ‘markers’ within a pedigree. Examples of such markers include restriction enzyme sites, visible chromosomal abnormalities such as translocations, single nucleotide polymorphisms and other mutations (e.g., microsatellite DNA, inversions, transversions, deletions, etc.). Relatively new fields such as proteomics and mRNA analysis (e.g., expression profiling) are also rapidly gaining in importance.

Epigenetic approaches. Additionally, a new and significant epigenetic field relating to DNA methylation pattern analysis is emerging. DNA methylation is the most common covalent modification of genomic DNA. The covalent attachment of a methyl group at the C5-position of the nucleotide base cytosine is particularly common within CpG dinucleotides of gene regulatory regions. The likelihood of finding any particular dinucleotide sequence in a given DNA sequence is 1/16 or ˜6%. In humans, however, the average genomic measured frequency of the CpG dinucleotide is very low (about 1/70). However, contiguous genomic regions of between 300 bp and 3000 bp in length exist, where the occurrence of CpG dinucleotides is significantly higher than normal. These CpG-rich regions are referred to in the art as CpG ‘islands’ and represent about 1% of the genome.

Such CpG islands have primarily been observed in the 5′-region of genes, and more than 60% of human promoters are contained in, or overlap with such CpG islands. Cytosine methylation within such CpG islands plays an important role in gene expression and regulation, in maintenance of normal cellular functions, and is associated with genomic imprinting and embryonic development. Furthermore, aberrant methylation patterns have been linked with a variety of disease conditions, and in particular with cancer. Many CpG islands are not in the promoters of genes, and their significance and function remains unclear.

Methylation assays. Various methods are currently used in the art for the analysis of specific CpG dinucleotide methylation status. These may be roughly characterized as belonging to one of two general categories: namely, restriction enzyme based technologies, or unmethylated cytosine conversion based technologies.

Restriction enzyme based technologies. The use of methylation sensitive restriction endonucleases for the differentiation between methylated and unmethylated cytosines is perhaps the oldest, and most widely-recognized technique. Restriction enzymes characteristically hydrolyze (cleave) DNA at and/or upon recognition of specific sequences (i.e., recognition motifs) that are typically between 4- to 8-bases in length. Among such enzymes, methylation sensitive restriction enzymes are distinguished by the fact that they either cleave, or fail to cleave DNA according to the cytosine methylation state present in the recognition motif (e.g., the CpG sequences thereof).

In methods employing such methylation sensitive restriction enzymes, the digested DNA fragments are typically separated (e.g. by gel electrophoresis) on the basis of size, and the methylation status of the sequence is thereby deduced, based on the presence or absence of particular fragments. Preferably, a post-digest PCR amplification step is added wherein a set of two oligonucleotide primers, one on each side of the methylation sensitive restriction site, is used to amplify the digested DNA. PCR products are not detectable where digestion of the subtended methylation sensitive restriction enzyme site occurs.

The applicability of this technique, in many cases, is limited by the few species of enzymes available and the distribution of their corresponding recognition motifs. Furthermore, these techniques are costly, time consuming, and result in the analysis of only individual sites per reaction. Nonetheless, restriction enzyme based technologies have proven utility for genome-wide assessments of methylation patterns, particularly where sequence data is unavailable. Techniques for restriction enzyme based analysis of genomic methylation include the following: differential methylation hybridization (DMH) (Huang et al., Human Mol. Genet. 8, 459-70, 1999); Not I-based differential methylation hybridization (see e.g., WO 02/086163 Al); restriction landmark genomic scanning (RLGS) (Plass et al., Genomics 58:254-62, 1999); methylation sensitive arbitrarily primed PCR (AP-PCR) (Gonzalgo et al., Cancer Res. 57: 594-599, 1997); methylated CpG island amplification (MCA) (Toyota et. al., Cancer Res. 59: 2307-2312, 1999).

Cytosine conversion based technologies. A more common and utilitarian method of CpG methylation status analysis comprises methylation status-dependent chemical modification of CpG sequences within isolated genomic DNA, or within fragments thereof, followed by DNA sequence analysis. Chemical reagents that are able to distinguish between methylated and non methylated CpG dinucleotide sequences include hydrazine, which cleaves the nucleic acid, and the more preferred bisulfite treatment. Bisulfite treatment followed by alkaline hydrolysis specifically converts non-methylated cytosine to uracil, leaving 5-methylcytosine unmodified (Olek A., Nucleic Acids Res. 24:5064-6, 1996). The bisulfite-treated DNA may then be analyzed by conventional molecular biology techniques, such as PCR amplification, sequencing, and detection comprising oligonucleotide hybridization.

Herman and Baylin first described the use of methylation-sensitive primers for the analysis of CpG methylation status with isolated genomic DNA (Herman et al. Proc. Natl. Acad. Sci. USA 93:9821-9826, 1996, and by U.S. Pat. No. 5,786,146; see also U.S. Pat. No. 6,265,171). The described method, methylation sensitive PCR (MSP), allows for the detection of a specific methylated CpG position within, for example, the regulatory region of a gene. The DNA of interest is treated such that methylated and non-methylated cytosines are differentially modified (e.g., by bisulfite treatment) in a manner discernable by their hybridization behavior. PCR primers specific to each of the methylated and non-methylated states of the DNA are used in a PCR amplification. Products of the amplification reaction are then detected, allowing for the deduction of the methylation status of the CpG position within the genomic DNA.

Other methods for the analysis of bisulfite treated DNA include methylation-sensitive single nucleotide primer extension (Ms-SNuPE) (Gonzalgo & Jones, Nucleic Acids Res. 25:2529-2531, 1997; and see U.S. Pat. No. 6,251,594), and the use of real time PCR based methods, such as the art-recognized fluorescence-based real-time PCR technique MethyLight™ (Eads et al., Cancer Res. 59:2302-2306, 1999; U.S. Pat. No. 6,331,393 to Laird et al.; and see Heid et al., Genome Res. 6:986-994, 1996).

However, while the methylation assay methods described herein are useful for the determination of the methylation status of particular genomic CpG positions, and despite continued investigation of the association of diseases with genomic methylation status, the clinical application of methylation status as a disease marker or as the basis for treatments has not emerged.

Presently, there are no commercially available diagnostic and/or prognostic assays for the analysis of the methylation status CpG dinucleotide sequence positions as markers for disease or disease-related conditions. Significantly, this situation does not reflect any lack of potential for such markers and applications, but rather relates to the that fact that there are no known systematic methods for the efficient identification, assessment and validation of such markers.

Therefore, there is a pronounced need in the art for a systematic method for the efficient identification, assessment and validation of differentially methylated genomic CpG dinucleotide sequences as diagnostic and/or prognostic markers.

### SUMMARY OF THE INVENTION

The subject matter of the present invention is directed, inter alia, to a method for the identification of methylated CpG dinucleotides within genomic DNA that may be used as clinically relevant markers. Said method comprises: a) formulating of a diagnostic aim of the marker; b) obtaining test and control samples; c) analyzing the samples by means of methods capable of identifying differentially methylated CpG dinucleotide sequences within the entire genome or a representative fraction thereof; d) further investigating the identified CpG positions of interest by analyzing the surrounding sequence context to further characterize the methylation patterns of the genomic region in question; e) further analyzing the identified or surrounding differentially methylated CpG positions within larger sample sets by using a methodology suitable for medium and/or high throughput comparison/screening, wherein the identified or surrounding CpG marker positions are analyzed by statistical means to identify reliable diagnostic and/or prognostic marker CpG positions.

Preferably, analyzing in c) comprises analysis of the literature for identification of CpG positions which may be of particular interest with respect to the formulated diagnostic aim, and optionally comprises relative scoring of the identified CpG positions to facilitate selecting the most promising identified candidate CpG marker positions for further analysis. Preferably, further investigating in d) comprises a scoring procedure to facilitate selecting a limited subset of the identified markers for further analysis. In a preferred embodiment, the method is implemented in a clinical or laboratory setting.

In alternate embodiments, the present invention provides a method for the identification of a reliable diagnostic and/or prognostic methylation marker within genomic DNA, comprising:

a) formulating a diagnostic aim for a methylation marker;

b) obtaining a biological sample from a test subject comprising subject genomic DNA;

c) identifying a primary differentially methylated CpG dinucleotide sequence of the test subject genomic DNA using a controlled assay suitable for identifying at least one differentially methylated CpG dinucleotide sequences within the entire genome, or a representative fraction thereof;

d) identifying, within a genomic DNA context region surrounding or including the primary differentially methylated CpG dincleotide, and using an assay suitable therefore, a secondary differentially methylated CpG dinucleotide sequence, or a pattern having a plurality of differentially methylated CpG dinucleotide sequences including the primary and at least one secondary differentially methylated CpG dinucleotide sequences; and

e) comparing, among a plurality of test genomic DNA samples corresponding to different test subjects, and using at least one of a medium- or a high-throughput controlled assay suitable therefore, the methylation states corresponding to the secondary differentially methylated CpG dinucleotide sequence, or to the pattern, whereby a reliable methylation marker is provided.

Preferably, identifying a primary differentially methylated CpG dinucleotide sequence in c) comprises analysis of the literature for identification of CpG positions which may be of particular interest with respect to the formulated diagnostic aim, and optionally comprises relative scoring of the identified CpG positions to facilitate selecting the most promising primary CpG marker position, or positions, for further analysis. Preferably, identifying a secondary differentially methylated CpG dinucleotide sequence, or a pattern having a plurality of differentially methylated CpG dinucleotide sequences in d) comprises a scoring procedure to facilitate selecting a limited subset of identified secondary differentially methylated CpG dinucleotide sequences, or patterns for further analysis. Preferably, the method is implemented in a clinical or laboratory setting.

### BRIEF DESCRIPTION OF THE DRAWINGS

FIG. 1 shows, in schematic form, components of a method according to the present invention.

FIG. 2 illustrates basic principles of methylation sensitive enzyme-mediated genome-wide methylation analysis methodologies.

FIG. 3 shows representative visual output formats of four different art-recognized genome-wide methylation analysis techniques, wherein differential methylation sites are identified by the presence or absence of bands of DNA, or hybridization intensity of spots (DMH). The techniques, from left to right are: Arbitrarily primed-PCR (AP-PCR); Methylated CpG island amplification (MCA); Restriction landmark genomic scanning (RLGS); and Differential methylation hybridization (DMH; also known as ECIST in particular embodiments).

FIG. 4 shows the polymerase mediated amplification of a CpG-rich sequence using methylation specific primers on four representative bisulfite-treated DNA strands (example cases “A”-“D”) (“MSP Amplification”). The methylation specific forward and reverse primers (“1”), in each case, can anneal to the bisulfite-treated DNA strand (“3”) if the corresponding subject genomic CpG sequences were methylated. The bisulfite-treated DNA strand (“3”) can be amplified if both forward and reverse primers (“1”) anneal, as shown in representative case “A” at the top of the figure.

FIG. 5 shows polymerase-mediated amplification analysis of bisulfite-treated DNA (“3”) corresponding to a CpG-rich genomic sequence by means of the MethylHeavy™ technique. Amplification of the treated DNA (“3”) is precluded if the blocking oligonucleotide (“5”) anneals to the treated DNA as shown for the example case “B.”

FIG. 6 shows the analysis of bisulfite-treated DNA using a MethyLight™ assay according to step 5 of the Example disclosed herein below. The Y-axis shows, using a log-scale, the percentage of methylation at the CpG positions covered by the corresponding CpG-specific probes. The dark bar (“A”) corresponds to tumor samples, whereas the white bar (“B”) correspond to healthy control tissue samples.

FIG. 7 shows the inventive differentiation of healthy tissue from non healthy tissue wherein the non healthy specimens are obtained from either colon adenoma or colon carcinoma tissue. The evaluation is carried out using informative CpG positions from 27 genes. Informative genes are further described in Table 4 herein below.

FIG. 8 shows the inventive differentiation of healthy colon tissue from carcinoma tissue using informative CpG positions from 15 genes. Informative genes are further described in Table 4 herein below.

FIG. 9 shows the inventive differentiation of healthy colon tissue from adenoma tissue using informative CpG positions from 40 genes. Informative genes are further described in Table 4 herein below.

FIG. 10 shows the inventive differentiation of colon carcinoma tissue from colon adenoma tissue using informative CpG positions from 2 genes. Informative genes are further described in Table 4 herein below.

FIG. 11 shows the sequence analysis of MeST number 15633, by sequencing of the pooled colon carcinoma samples. The upper trace, for each sequence region, shows the sequencing output prior to processing, the lower trace shows the trace post-processing.

FIG. 12 shows the sequencing analysis of specific CpG positions of MeST number 15633, within individual samples. Each horizontal line represents a specific CpG site. Each vertical column represents a different sample. Blue stands for a methylated status and yellow for an unmethylated status. Intermediate status are represented by shades of green. Failures are represented by white fields.

FIG. 13 shows the amplification of bisulphite-treated DNA according to Step 5 of the Example disclosed herein below. The lower trace (“B”) shows the amplification of DNA from normal colon tissue, while the upper trace (“A”) shows the amplification of DNA from tumor tissue. The X-axis shows the cycle number of the amplification, whereas the Y-axis shows the amount of amplificate detected.

FIG. 14-shows an analysis of bisulphite-treated DNA using the combined HeavyMethyl™ MethyLight™ assay according to Step 5 of the Example disclosed herein below. The Y-axis shows, using log scale, the percentage of methylation at the CpG positions covered by the probes. The dark bar corresponds to tumor samples, whereas the white bar corresponds to normal control tissues.

### DETAILED DESCRIPTION OF THE INVENTION

The present invention provides, in particular embodiments, a systematic method for the efficient identification, assessment and validation of differentially methylated genomic CpG dinucleotide sequences as diagnostic and/or prognostic markers.

Definitions:

The term “Observed/Expected Ratio” (“O/E Ratio”) refers to the frequency of CpG dinucleotides within a particular DNA sequence, and corresponds to the [number of CpG sites/(number of C bases×number of G bases)]×band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length, and may be as large as about 3 Kb in length.

The term “methylation state” or “methylation status” refers to the presence or absence of 5-methylcytosine (“5-mCyt”) at one or a plurality of CpG dinucleotides within a DNA sequence. Methylation states at one or more particular palindromic CpG methylation sites (each having two CpG dinucleotide sequences) within a DNA sequence include “unmethylated,” “fully-methylated” and “hemi-methylated.”

The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CC^{M}GG-3′ (top strand): 3′-GGCC-5′ (bottom strand)).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to a HeavyMethyl™ MethylLight™ assay, which is a variation of the MethylLight™ assay, wherein the MethylLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401 A1.

The term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The phrase “sequence context of selected CpG dinucleotide sequences” refers to a genomic region of from 2 nucleotide bases to about 3 Kb surrounding or including a primary differentially methylated CpG dinucleotide identified by the genome-wide Discovery methods described herein (in Step 2 of the inventive method). Said context region comprises, according to the present invention, at least one secondary differentially methylated CpG dinucleotide sequence, or comprises a pattern having a plurality of differentially methylated CpG dinucleotide sequences including the primary and at least one secondary differentially methylated CpG dinucleotide sequences. Preferably, the primary and secondary differentially methylated CpG dinucleotide sequences within such context region are comethylated in that they share the same methylation status in the genomic DNA of a given tissue sample. Preferably the primary and secondary CpG dinucleotide sequences are comethylated as part of a larger comethylated pattern of differentially methylated CpG dinucleotide sequences in the genomic DNA context. The size of such context regions varies, but will generally reflect the size of CpG islands as defined above, or the size of a gene promoter region, including the first one or two exons.

Unless defined otherwise, all technical and scientific terms used herein have the same meaning as commonly understood by one of ordinary skill in the art to which the invention pertains. Although any methods and materials similar or equivalent to those described herein can be used for testing of the present invention, the preferred materials and methods are described herein. All documents cited herein are thereby incorporated by reference.

### A Systematic Method for the Efficient Identification of Reliable Diagnostic and/or Prognostic Methylation Markers Within Genomic DNA

The present invention provides a systematic method for the efficient identification, assessment and validation of differentially methylated genomic CpG dinucleotide sequences as diagnostic and/or prognostic markers.

The present invention is directed to a method for the identification of differentially methylated CpG dinucleotides within genomic DNA that are particularly informative with respect to disease states. These may be used either alone or as components of a gene panel in diagnostic and/or prognostic assays.

In particular embodiments, the invention is directed to the identification of CpG positions which may be used as markers for the diagnosis or prediction of unwanted side effects of medicaments, and of disease and disease-related conditions, including but not limited to: cell proliferative disorders, such as cancer; dysfunctions, damages or diseases of the central nervous system (CNS), including aggressive symptoms or behavioural disorders; clinical, psychological and social consequences of brain injuries; psychotic disorders and disorders of the personality, dementia and/or associates syndromes; cardiovascular diseases, malfunctions or damages; diseases, malfunctions or damages of the gastrointestine diseases; malfunctions or damages of the respiratory system; injury, inflammation, infection, immunity and/or reconvalescence, diseases; malfunctions or damages as consequences of modifications in the developmental process; diseases, malfunctions or damages of the skin, muscles, connective tissue or bones; endocrine or metabolic diseases malfunctions or damages; headache; and sexual malfunctions; or combinations thereof.

Presently, there are no commercially available diagnostic and/or prognostic assays for the analysis of the methylation status of CpG dinucleotide sequence positions as markers for disease or disease-related conditions. Furthermore, and significantly, there are no known systematic methods for the identification, assessment and validation of such markers. The present invention provides such a systematic means for the identification and verification of multiple disease relevant CpG positions to be used alone, or in combination with other CpG positions (e.g, as a panel or array of markers), to form the basis of a clinically relevant diagnostic assay.

The inventive method enables differentiation between two or more phenotypically distinct classes of biological matter. Said method comprising the comparative analysis of the methylation patterns of CpG dinucleotides within each of said classes. Said method comprising the following steps 1-4, and optionally, step 5:

Step 1: Definition of one or more phenotypic parameters that distinguish between or among at least two classes of biological samples to formulate a diagnostic aim for a methylation marker.

Step 2: Determination of differences in CpG methylation between said at least two classes of biological samples by means of analysis of the genome-wide methylation patterns of biological samples of both classes. Said analysis carried out by: (i) analysis of the methylation status of one or more CpG positions within each of said samples and/or classes; (ii) comparison of the methylation status of the analyzed CpG position(s) between each of said classes; and (iii) identification of the CpG positions differentially methylated between said classes. Thus, step 2 provides for identifying one or more primary differentially methylated CpG dinucleotide sequences of a test subject genomic DNA using a controlled assay suitable for identifying at least one differentially methylated CpG dinucleotide sequences within the entire genome, or a representative fraction thereof;

Step 3: Determination of the characteristic methylation patterns of CpG positions in the vicinity of the differentially methylated CpG positions identified in Step 2, and thereby determining further CpG positions differentially methylated between said classes. Thus, step 3 provides for identifying, within a genomic DNA ‘context’ region surrounding or including one or more primary differentially methylated CpG dincleotides, and using an assay suitable therefore, one or more secondary differentially methylated CpG dinucleotide sequences, or a pattern having a plurality of differentially methylated CpG dinucleotide sequences and including the primary and at least one secondary differentially methylated CpG dinucleotide sequences.

Step 4: Analyzing the methylation status of differentially methylated CpG positions identified in Step 3 within larger numbers of biological samples of each class and analyzing the data in order to identify CpG positions which are suitable for reliably distinguishing between said classes of DNA either singularly or in combination with other CpG positions. Thus, step 4 provides for comparing, among a plurality of test genomic DNA samples corresponding to different test tissues and/or subjects, and using, preferably, at least one of a medium- or a high-throughput controlled assay suitable therefore, the methylation states corresponding to the secondary differentially methylated CpG dinucleotide sequence, or to the pattern, whereby a reliable methylation marker is provided.

The method may further comprise Step 5; the development of an assay for the analysis of the identified CpG marker positions.

Step 1—Experimental Design and Sample Collection:

In the step 1 of the inventive method, the diagnostic question to be addressed is formulated. The inventive method is used to compare two or more types of phenotypically distinct classes of samples (e.g., nucleic acids, genomes, cells, tissues, etc.). In principle, CpG methylation analysis is used for distinguishing cells, tissues or organisms which are otherwise genotypically identical or similar at the relevant genes, but are nonetheless phenotypically distinct.

The word ‘phenotype’ shall hereinafter be used to mean any observable and/or detectable characteristic of an organism or component thereof, where each characteristic may also be defined as a parameter contributing to the definition of the phenotype, and wherein a phenotype is defined by one or more parameters. An organism that does not conform to one or more of said parameters shall be defined to be distinct or distinguishable from organisms of said phenotype. In the inventive method, the diagnostic question is formulated such that two or more phenotypically distinct classes of biological matter (hereinafter also referred to as ‘classes’) are differentiated from one another. Parameters may either be continuous (e.g., age, survival time, etc.) or discontinuous (e.g., presence or absence of a disease).

In a preferred embodiment, the phenotypes are defined according to one or more parameters belonging to the following classes A-Q:

A) The presence, absence or characteristics of one or more diseases or their sub-types belonging to the following classes:

Cell proliferative disorders; metabolic malfunctions or disorders; immune malfunctions, damage or disorders; CNS malfunctions, damage or disease; symptoms of aggression or behavioural disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, the muscles, the connective tissue or the bones; endocrine and metabolic malfunction, damage or disease; headaches or sexual malfunction.

B) Disease diagnosis; detailed parameters such as blood pressure, cancer staging, sugar levels etc.; C) Pharmacological treatment and/or treatment response; D) Age; E) Life style; F) Disease history; G) Molecular biological parameters (e.g., signaling chains and protein synthesis); H) Behavior; I) Drug abuse; J) Patient history; K) Cellular parameters; L) Histological parameters; M) Physiological parameters; N) Anatomical parameters; O) Pathological parameters; P) Treatment history; and Q) Gene expression.

For example, in one embodiment of the method, patients over 60-years old having Grade-1 carcinoma of the prostate peripheral zone, are distinguished from those over 60-years old having benign prostate hyperplasia, wherein said patients have comparable medical histories and life styles.

The question to be formulated should be clinically relevant, technically feasible and preferably commercially significant in having a significant market size for the diagnostic assay. For example the method according to the invention as described herein may be used for the development of diagnostic tools for the grading and staging of cancers, for use in prenatal diagnosis, and for the detection of a predisposition to a variety of methylation related diseases.

A preferred method according to the invention is characterized in that the at least one phenotypic class is derived from biological material of diseased individuals and in subsequent steps of the method compared to biological material of healthy individuals. Such diseases include all diseases and/or medical conditions which involve a modification of the expression of cellular genes and include, for example: unwanted side effects of medicaments; cancers, metastasis; dysfunctions, damages or diseases of the central nervous system (CNS); aggressive symptoms or behavioural disorders; clinical, psychological and social consequences of brain injuries; psychotic disorders and disorders of the personality, dementia and/or associates syndromes; cardiovascular diseases; malfunctions or damages, diseases, malfunctions or damages of the gastrointestine; diseases, malfunctions or damages of the respiratory system; injury, inflammation, infection, immunity and/or reconvalescence, diseases; malfunctions or damages as consequences of modifications in the developmental process; diseases, malfunctions or damages of the skin, muscles, connective tissue or bones; endocrine or metabolic diseases, malfunctions or damages; headache; sexual malfunctions; leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease; or combinations thereof.

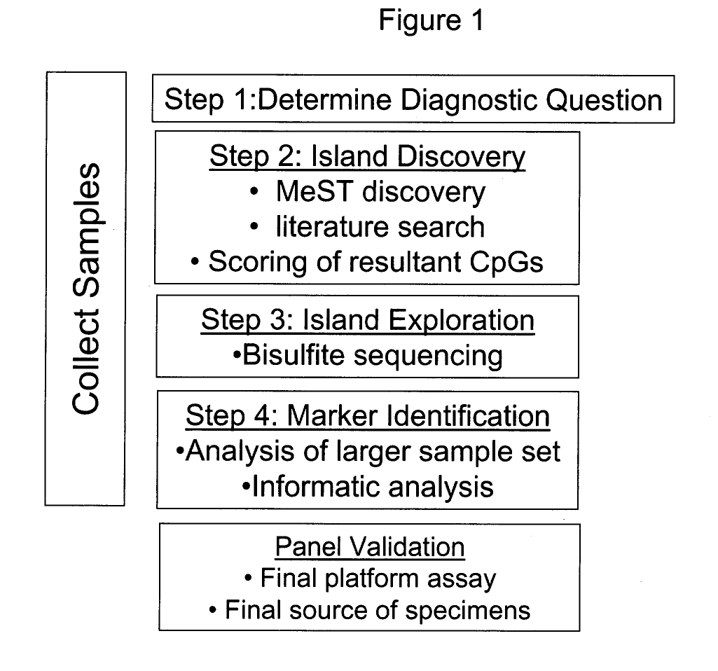

In a preferred embodiment of the method, subsequent to the formulation of the diagnostic aim of the marker suitable biological samples are sourced and acquired. Sourcing and acquisition of the samples may be completed prior to the initiation of the next step (Step 2) or in a preferred embodiment of the method sourcing and acquisition of the samples may be ongoing with subsequent steps of the method (see FIG. 1).

Samples may be obtained according to standard techniques from all types of biological sources that are usual sources of DNA including, but not limited to cells or cellular components which contain DNA, cell lines, biopsies, bodily fluids such as blood, sputum, stool, urine, cerebrospinal fluid, ejaculate, tissue embedded in paraffin such as tissue from eyes, intestine, kidney, brain, heart, prostate, lung, breast or liver, histological object slides, and all possible combinations thereof.

Samples should be representative of the target population and should be as unbiased as possible. Steps 2 and 3 of the method require obtaining genomic DNA from a high-quality source (e.g., said sample should contain only the tissue type of interest, minimum contamination and minimum DNA fragmentation). During Step 4, samples should be representative of the type that is to be handled by the diagnostic assay (i.e., may be of less pure quality) and samples are analyzed individually rather than pooled. Preferably, during Steps 2 and 3, each class to be analyzed should be represented by a sample set size of 10 or above. Preferably, for Step 4, analysis is carried out on sample set sizes in the hundreds.

In subsequent steps of the method, the methylation levels of CpG positions are compared between the at least two classes, to identify differentially methylated CpG positions. Each class may be further segregated into sets according to predefined parameters to minimize the variables between the at least two classes. In the following stages of the method, all comparisons of the methylation status of the classes of tissue, are carried out between the phenotypically matched sets of each class. Examples of such variables include, age, ethnic origin, sex, life style, patient history, drug response etc.

Step 2—CpG Island Discovery:

Once suitable sets of tissue samples have been established (e.g., number of samples being 10 or more, all of high quality, and in a preferred embodiment, the sample set consists of tester- and driver-matched pair samples for comparison), Step 2 of the method may be initiated. This step is herein also referred to as ‘CpG Island Discovery’ or simply ‘Island Discovery.’

The aim of this step of the method is to survey the entire genome for phenotypically characteristic CpG methylation patterns. CpG positions representative of a significant proportion of the genome are analyzed to ascertain the methylation status of the different classes on a genome-wide basis or level. The methylation pattern of each sample set is characterized and CpG positions differentially methylated between the sets are identified. In a preferred embodiment, at least 50 different CpG positions are analyzed, and in a particularly preferred embodiment the analyzed CpG positions are situated within at least 20 different discrete genes and or their promoters, introns, first exons and/or enhancers.

Step 2 identifies CpG positions relevant to the diagnostic/prognostic aim of interest by use of molecular biological methods, optionally supplemented by analysis of the published state of the art. The CpG positions which are identified as being differentially methylated between the sample sets and/or classes in this step of the method are termed ‘Methylated Sequence Tags’ or MeSTs.

Preferably, the methods used to characterize the methylation patterns of each sample set (hereinafter also referred to as ‘Discovery techniques’) enable a genome-wide methylation pattern analysis. In a particularly preferred embodiment, the characterization is carried out by means of methylation sensitive restriction enzyme digest analysis, and in particular by means of one or a combination of the following techniques: Methylated CpG island amplification (MCA); Arbitrarily primed PCR (AP-PCR); Restriction landmark genomic scanning (RLGS); Differential methylation hybridization (DMH, also known as ECIST); and NotI restriction based differential hybridization method.

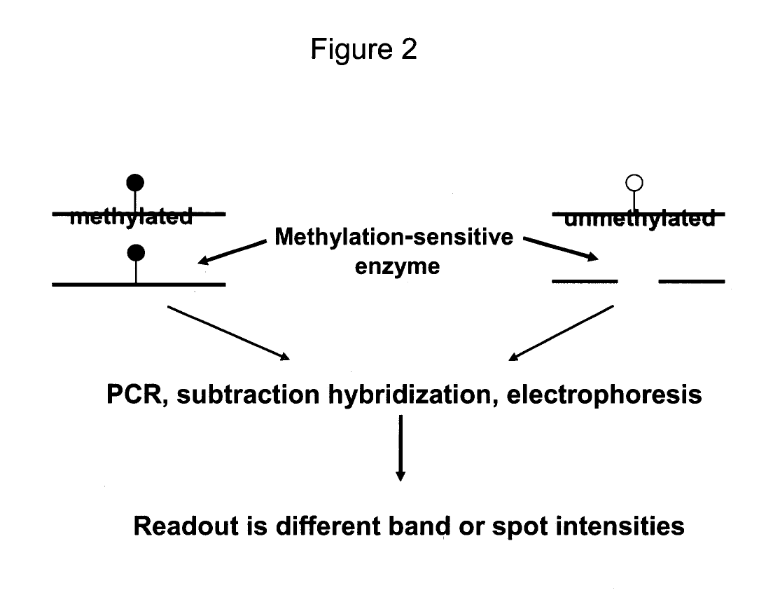

An overview of the basic principle of the methylation sensitive enzyme based methodologies is shown in FIG. 2.

A more detailed explanation of some of the preferred Discovery techniques follows:

Differential methylation hybridization (DMH). DMH is a microarray compatible approach that simultaneously detects DNA methylation in thousands of CpG islands. The first part of DMH is the generation of multiple CpG island tags (CGI library) as templates arrayed onto solid supports (e.g., glass slides or nylon membranes). The generation of CpG island tags has been described (Huang et al., Human Mol. Genet. 8, 459-70, 1999). Briefly, genomic DNA is isolated, purified and digested using a restriction enzyme that is unlikely to digest within CpG islands, for example MseI (TTAA). The DNA digest is then enriched for CpG-rich regions (e.g., by in vitro methylation of the digest and purification using a methylated DNA binding column consisting of a polypeptide of the DNA binding domain of the rat MeCP2 protein attached to a solid support; as described by Cross et. al Nature Genetics 6:236-244, 1994). The restriction fragments are screened for repeat elements and PCR amplified. The fragments are then fixed in the form of an array on a solid surface (e.g., glass slide, nylon membrane), in a manner whereby each fragment is locatable and identifiable on the surface.

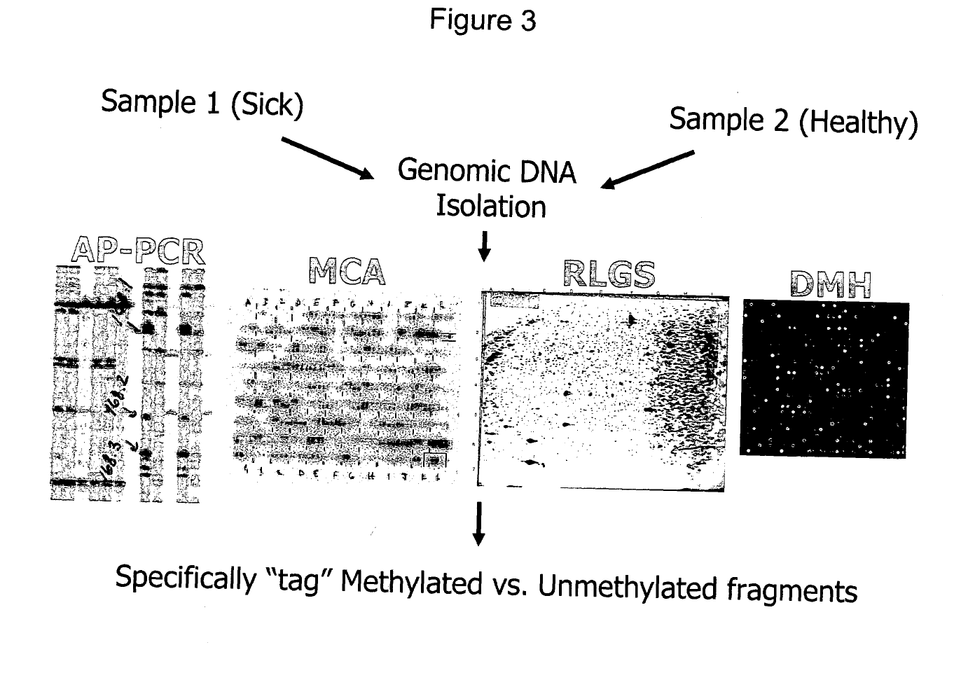

The second part involves preparation of amplicons, corresponding to test and reference (control) genomes. Amplicons are used as probes in array-hybridization. Briefly, for amplicon generation, genomic DNA from both the test and reference samples are isolated. Each DNA sample is digested using an enzyme unlikely to digest within CpG islands (e.g., the same enzyme as was used to generate the CGI library). Linker sequences are ligated to the ends of the DNA fragments, and the DNA fragments digested using one or more methylation sensitive restriction enzymes. The digest fragments are PCR amplified and labeled. No PCR amplificate is detectable where the restriction of a fragment has taken place during the second digest. The labeled PCR products are hybridized to the CGI library generated earlier. Comparison of the hybridization pattern of PCR fragments from different types of tissues allows for the detection of differences in methylation patterns between the two types of tissues (see FIG. 3). Positive signals identified by the test amplicon, but not by the reference amplicon, indicate the presence of hypermethylated CpG island loci in test cells.

Restriction landmark genomic scanning (RLGS). In RLGS-based methods, differential methylation of CpG positions is discriminated based on digestion of genomic DNA with a methylation sensitive restriction endonuclease. RLGS provides quantitative analysis of CpG islands separated by two-dimensional gel electrophoresis into discrete spots. The resulting spot patterns, or RLGS profiles, are highly reproducible, and thus amenable to intra- and inter-individual comparison.

In a particularly preferred embodiment, each sample is analyzed as a member of a paired set for comparison. DNA is extracted using standard methods known in the art (e.g., by using commercially available kits). Each sample is treated (cleaved ends and nicks and gaps are filled with nucleotide analogues) to prevent random labeling of the DNA strands. Blocking the random (sheared) ends of the whole genomic DNA in the initial DNA preparations for RLGS include the addition of modified nucleotide bases to overhanging ends, where the newly added nucleotides prevent addition of other bases (radio-labeled nucleotides) in later steps. The modified nucleotides are a mixture of dideoxy-ATP, dideoxy-dTTp, dGTP-alpha-S & dCTP-alpha-S. The nucleotides are added to the overhanging ends with standard techniques using either DNA Polymerase 1 or Klenow enzyme (see e.g., Hatada et al., Proc Natl Acad. Sci. U S A. 88:9523-7, 1991).

The treated DNA is digested using a landmark restriction enzyme, for example but not limited to, NotI. The restriction enzyme is deactivated and the digest fragments are labeled at the restriction site. Cleaved landmark restriction sites are preferably labeled with a radioisotope. The genomic DNA is further fragmented, in a progressive manner, with restriction endonucleases with sequence recognition specificity that does not recognize sequences containing CpG, to separate the CpG islands.

For two purposes of dimensional separations, the digest fragments are separated by size, for example by using a high-resolution gel electrophoresis in a first dimension. The nucleic acid fragments are subjected to a restriction enzyme digest carried out in the gel. After digestion, the fragments are electrophorized a second time with the current running perpendicular relative to the direction of the current in the first electrophoresis. Each gel is exposed using X-ray film or other such suitable methods compatible with the detectable label used to produce a fixed image of the positions of the fragment within the gel (see FIG. 3). The highly reproducible DNA fragment patterns on the x-ray films exposed to each of the 2-dimensional gels (referred to as “RLGS Profiles”) are then compared to determine where the patterns differ.

Methylation-Sensitive Arbitrarily-Primed Polymerase Chain Reaction (MS.AP-PCR). MS.AP-PCR refers to the art-recognized technology that allows for a global scan of the genome using CG-rich primers to focus on the regions most likely to contain CpG dinucleotides, and described by Gonzalgo et al., Cancer Research 57:594-599, 1997. For present inventive applications of MS.AP-PCR methods, the two classes of DNA samples are each-digested with at least one species of restriction endonuclease, of which at least one is a methylation sensitive restriction endonuclease. The digested fragments are amplified in a PCR reaction of variable stringency, as determined by the investigator. At least one of the primers used in the amplification reaction is/are arbitrarily designed. PCR amplificates from both test and driver samples are compared to identify CpG positions differentially methylated between the test and driver classes (see FIG. 3).

Methylated CpG island amplification (MCA). MCA is based on sequential restriction enzyme digestion with methylation-sensitive/insensitive isoschizomers, adaptor ligation and whole-methylated-genome PCR. A first digestion is carried out upon the genomic DNA of interest using a methylation sensitive restriction enzyme (e.g., SmaI). SmaI is a methylation sensitive restriction enzyme that does not cut when its recognition sequence CCCGGG contains a methylated CpG position, whereas unmethylated CpG positions are digested leaving blunt edged fragments. The SmaI digest is redigested using the methylation insensitive isoschizomer of the enzyme used previously, said digestion leaving sticky ends. For example, SmaI digests are digested by use of the SmaI isoschizomer XmaI, which leaves a sticky edged CCGG overhang. Adaptors are then ligated to the sticky ends and the fragments are amplified, preferably by means of PCR. The amplificate fragments may then be analyzed using a number of methods (e.g., chromatographic methods, sequencing, hybridization analysis) for analysis and comparison of methylation status both within and between classes of tissue. In a preferred embodiment of the method, said analysis is carried out by hybridization of the test to the driver amplificates and subtraction of the fragments common to both.

FIG. 3 shows the different formats of the final results of the above-described Discovery methodologies. MeSTs which are differentially methylated between the two or more classes of tissues are identified by comparison of the restriction pattern or spots generated.

NotI restriction based differential methylation hybridization (NR-DMH). NR-DMH is another microarray compatible approach that simultaneously detects DNA methylation in a thousands of CpG islands. The first part of NR-DMH involves generation of a NotI flanking clone library, containing multiple clones specified by consisting of pairs of sequences flanking a single NotI recognition site. To generate these clones, which contain nucleic acid bases 5′ and 3′ of the NotI restriction site, genomic DNA is isolated from a source having a low level of methylation. In a preferred embodiment, the genomic DNA is isolated from any human cell and in an additional step demethylated before generating the clones. The DNA is purified and digested using a restriction enzyme that is likely to cut within the proximity of NotI sites and leaves sticky ends with the fragment. In a preferred embodiment, these enzymes are BamHI and BgII. The digests are diluted and then circularized by catalyzing their self-ligation. These circularized clones are treated with the restriction enzyme NotI, which cuts only if the CpG sites at the restriction site is unmethylated. These clones are arrayed onto solid supports (e.g., glass slides or nylon membranes), in a manner whereby each clone is locatable and identifiable on the surface.

Labeled fragments, representing pooled DNA from the test and reference (control) genomes, are next prepared. Said fragments are used as probes in the array-hybridization step. Positive signals identified by the reference fragment, but not by the test fragment, indicate the presence of hypermethylated CpG sites in the test cells.

Briefly, genomic DNA from both the test and reference samples are isolated. Each DNA sample is then digested using an enzyme unlikely to digest within CpG islands, the same enzyme or combination of enzymes as was used to generate the NotI flanking clone library. Again these digests are diluted and the fragments self-ligated. Subsequently, the circularized clones are digested with the restriction enzyme NotI. NotI will not cut where methylated cytosines occur in the restriction site. The linearized DNA is PCR amplified, labeled and hybridized to the chip.

In a preferred embodiment, after the NotI restriction digest is stopped, NotI restriction site specific linker sequences are ligated to the ends of the DNA fragments. In the next step these linkers provide the specific priming sites for primer oligonucleotides during a PCR amplification. It is also preferred that the PCR is a ‘hot’ PCR to avoid a separate step of labeling the amplicons.

Where linearization of a circularized fragment has not taken place during the NotI digest, no PCR amplificate is detectable. The labeled PCR products are then hybridized to the NotI flanking clone library generated earlier. Comparison detection of differences in methylation patterns between the two types of tissues.

In a preferred embodiment of the inventive method, Step 2 is supplemented by a literature search of all published art; including genome databases and peer-reviewed publications of the art, to identify CpG positions of relevance to the diagnostic and/or prognostic aim. The two groups of CpG positions thus identified, are combined.

In a particularly preferred embodiment of the inventive method, the candidate marker CpG positions are further assessed by using a scoring system to rank MeSTs according to their potential as marker candidates for progression to Step 3 of the method (see FIG. 3):

Scoring. Investigation of all candidate differentially methylated CpG positions identified is likely to be unproductive and costly. Therefore, in a particularly preferred embodiment of the method, subsequent to steps 2 and 3 of the method each candidate CpG position is scored as to its suitability for further analysis. Scoring parameters include, but are not limited to the following parameters, or a combination thereof:

Confirmation of the MeST; that is, has it been possible to identify the MeST using only one technique, or has it been possible to verify its differential methylation status using multiple techniques?;

Tissue specificity; that is, has the same MeST shown up in different classes of tissues, and if so, was this achieved using the one method or multiple methods?;

Sequence context; that is does the CpG position occur in an area indicating that it may be of further interest (e.g., within a CpG island or close to a gene that has been already identified as a marker (both positive) or does it occur within microsatellite DNA (negative)).

Gene association; that is, if the MeST is associated with a gene, where is its location (e.g., promoter region, coding region, Intron or 3′-region); MeSTs within the 5′-promoter region are the most suitable candidates for further investigation; and

Association with an implicated gene; that is, if the MeST is associated with a gene, does the associated gene have known functional or etiological relevance (e.g., if the test tissue was neoplastic tissue, genes that are associated with transcription factors, growth factors, tumor suppressors or oncogenes would score highly).

Thus, step 2 provides a method for identifying one or more primary differentially methylated CpG dinucleotide sequences of a test subject genomic DNA using a controlled assay suitable for identifying at least one differentially methylated CpG dinucleotide sequences within the entire genome, or a representative fraction thereof.

Step 3—Investigation of Sequence Context of Selected CpG Dinucleotide Sequences:

The techniques used in Step 2 of the method allow for the identification of particular CpG positions of interest without providing information about the methylation patterns of the sequence context in which they occur. In Step 3 of the method, the sequence context of the MeSTs are investigated to ascertain methylation patterns of one or more surrounding CpG dinucleotide sequences. CpG positions occurring in CpG-rich islands of the genome are often co-methylated (wherein a significant proportion of the CpG positions within the island share the same methylation status). It is particularly preferred that marker positions occur in co-methylated islands to enable easier assay development (see Step 5).

The phrase “sequence context of selected CpG dinucleotide sequences” refers, for purposes of the present invention, to a genomic region of from 2 nucleotide bases to about 3 Kb surrounding or including a primary differentially methylated CpG dinucleotide identified by the genome-wide Discovery methods described herein (in Step 2 of the inventive method). Said context region comprises, according to the present invention, at least one secondary differentially methylated CpG dinucleotide sequence, or comprises a pattern having a plurality of differentially methylated CpG dinucleotide sequences including the primary and at least one secondary differentially methylated CpG dinucleotide sequences. Preferably, the primary and secondary differentially methylated CpG dinucleotide sequences within such context region are comethylated in that they share the same methylation status in the genomic DNA of a given tissue sample. Preferably the primary and secondary CpG dinucleotide sequences are comethylated as part of a larger comethylated pattern of differentially methylated CpG dinucleotide sequences in the genomic DNA context. The size of such context regions varies, but will generally reflect the size of CpG islands as defined above, or the size of a gene promoter region, including the first one or two exons.

Analysis of the sequence context of the MeSTs is generally taken, in the case of inventive gene associated CpG sequences, to be sequence analysis of the promoter and first exon regions of associated genes, and/or the CpG island within which the MeST lies, but this is left to the discretion of a person skilled in the art.

Said analysis may be carried out by any means known in the art (e.g., restriction enzyme based technologies, probe hybridization etc.), however, in the most preferred embodiment of the method said step is carried out by means of bisulfite treatment of the genomic DNA followed by sequencing.

The procedure that is described here is based on the bisulfite-dependent modification of all non-methylated cytosines to uracil, which exhibits the same base pairing behavior as thymine. Sodium bisulfite reacts with the 5,6-double bond of cytosine, but not with methylated cytosine. Cytosine reacts with the bisulfite ion to form a sulfonated cytosine reaction intermediate, which is susceptible to deamination, giving rise to a sulfonated uracil. The sulfonate group can be removed under alkaline conditions, resulting in the formation of uracil. Uracil is recognized as a thymine by polymerase and thereby upon PCR, the resultant product contains cytosine only at the position where 5-methylcytosine occurs in the starting template DNA. Thus, in DNA treated with bisulfite, 5-methylcytosine can easily be detected by virtue of its hybridization to guanine. This enables the use of variations of established methods of molecular biology, such as sequencing. Sequencing of bisulfite-treated DNA has been described (see e.g., Grunau C, et al., Nucleic Acids Res. 29:E65-5, 2001).

Sequencing of the bisulfite-treated DNA may be carried out using any technique standard in the art, such as the Maxam-Gilbert method and other methods such as sequencing by hybridization (SBH), but is most preferably carried out using the Sanger method. Primer selection is crucial in bisulfite based methylation analysis, since the complexity of DNA is reduced (unless methylation is present, there are only 3 bases on the strand). It is preferred that said primers be designed such that they do not contain any CG dinucleotide. Furthermore, in a preferred embodiment of the method, they are analyzed for specificity by testing them on genomic DNA (where no amplificates should be obtained).

A further preferred embodiment employs the cycle-sequencing method, also called linear amplification sequencing (see e.g., Stump et al., Nucleic Acids Res., 27:4642-8, 1999; Fulton & Wilson Biotechniques 17:298-301, 1994). Like the standard PCR reaction, it uses a thermostable DNA polymerase and a temperature cycling format of denaturation, annealing and DNA synthesis. The difference is that cycle sequencing employs only one primer and includes a ddNTP chain terminator in the reaction. The use of only a single primer means that unlike the exponential increase in product during standard PCR reactions, the product accumulates in a linear manner. Because the product accumulates during the reaction, and because of the high temperature at which the sequencing reactions are carried out, and the multiple heat denaturation stages, small amounts of double stranded plasmids, cosmids and PCR products may be sequenced reliably without a separate heat denaturation step.

In a further embodiment of the inventive method, samples of DNA are pooled with other members of their class thereby requiring only one sequencing reaction per class. Subsequent to sequencing it may be apparent that both methylated and unmethylated versions of each CpG position are detected within a class thereby allowing an assessment of the degree of methylation of a CpG position within a specific class.

In a preferred embodiment of the method, unsuitable candidate marker CpG positions may be eliminated by means of a scoring system (as carried out in Step 2) subsequent to sequencing of bisulfite-treated DNA. It is particularly preferred that CpG positions not exhibiting co-methylation (methylation of multiple CpG positions) within the examined ‘contex’ region are not analyzed in the subsequent steps of the inventive method.

Thus, step 3 provides for identifying, within a genomic DNA ‘context’ region surrounding or including one or more primary differentially methylated CpG dincleotides, and using an assay suitable therefore, one or more secondary differentially methylated CpG dinucleotide sequences, or a pattern having a plurality of differentially methylated CpG dinucleotide sequences and including the primary and at least one secondary differentially methylated CpG dinucleotide sequences.

Step 4—Marker Identification:

Step 4, also referred to as the Marker Identification Step, is carried out subsequent to sequencing of bisulfite-treated DNA and scoring. As many samples as possible from all classes of tissue analyzed during Steps 2 and 3, as well as any further classes of tissues that may wish to be compared should be analyzed in Step 4. The total number of samples should ideally be in the hundreds. Typically around 500 individual CpG positions may be investigated with an aim of reducing these to the 5-25 best markers for use singly or in the form of a panel.

Step 4 is carried out in two stages.

In Stage I, molecular biological techniques are used to analyze the methylation status of CpG positions identified in the previous steps (2 and 3). The methylation analysis is performed upon a sample set of increased size relative to that prior Steps 2 and 3. Such analysis may be carried out by several methods having versatility and mediuni/high throughput (e.g., parallel MS SNuPE). In a particularly preferred embodiment, however, the analysis is carried out by means of bisulfite-treatment followed by oliogonucleotide hybridization analysis using an array-based format.

Stage II of the Marker Identification Step is based on statistical and in silico analysis. In Stage II, the methylation status of each CpG position is assessed by statistical means as to its capability of discriminating between the DNA of the sample classes. CpG positions, which show significant methylation status differences between the classes are then combined to form a panel. Once the panel is defined, algorithmic methods for the classification of a sample, based on the methylation status of the panel CpG positions is developed. A suitable assay is thus developed in order to test the panel upon a larger sample set.

The two stages are explained in more detail herein below:

Stage I of Step 4. In a preferred embodiment of the method stage I of said Step 4 is carried out by means of hybridization analysis. In the most preferred embodiment, said analysis is carried out by means of the following steps:

In the first step of stage 1, the genomic DNA sample must be isolated from tissue or cellular sources. Such sources include, but are not limited to, cell lines, histological slides, bodily fluids or tissue embedded in paraffin. Extraction is by means that are standard to one skilled in the art, these include, but not limited to the use of detergent lysates, sonification, vortexing with glass beads, and precipitating with ethanol. Once the nucleic acids have been extracted and preferably purified, the genomic double-stranded DNA is used in the analysis.

In a preferred embodiment, the DNA may be cleaved prior to chemical treatment (below), by an art-recognized method, in particular with restriction endonucleases.

Subsequently, the genomic DNA sample is chemically treated in such a manner that cytosine bases, which are unmethylated at the C5-position are converted to uracil, thymine, or another base, which is detectably dissimilar to cytosine in terms of hybridization properties. This will be referred to hereinafter as ‘pretreatment,’ or, in particular embodiments, ‘bisulfite treatment.’

The above-described treatment of genomic DNA is preferably carried out with bisulfite (sulfite, disulfite) and subsequent alkaline hydrolysis, which results in conversion of non-methylated cytosine nucleobases to uracil, which is detectably dissimilar to cytosine in terms of base-pairing properties.

Fragments of the pretreated DNA are amplified, using sets of primer oligonucleotides and a polymerase. Preferably, the polymerase is a heat-stable polymerase. Preferably, because of statistical and practical considerations, more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two oligonucleotides (a forward primer and a reverse primer) in each case identical to a sequence comprising about 18 contiguous nucleotides, or more, of the pretreated nucleic acid.

In a particularly preferred embodiment, said set of primer oligonucleotides includes at least one pair of oligonucleotides, wherein said pair includes one oligonucleotide primer which is reverse complementary to a segment of the pretreated sequence to be amplified, and another which is identical to another segment of the pretreated sequence to be amplified. In a particularly preferred embodiment, said segment is at least 18 bases long. Preferably, the primer oligonucleotides do not comprise any CpG dinucleotides.

In a preferred embodiment of the present invention, at least one primer oligonucleotide is bound to a solid phase during amplification. The different oligonucleotide and/or PNA-oligomer sequences can be arranged on a plane solid phase in the form of a rectangular or hexagonal lattice. Preferably, the solid phase surface is composed of silicon, glass, polystyrene, aluminum, steel, iron, copper, nickel, silver, or gold. Other materials, such as nitrocellulose or plastics also have utility as solid phases.

The fragments obtained by means of the amplification (also referred to herein as ‘amplificates’) can carry a directly or indirectly detectable label. Preferred are labels in the form of fluorescence labels, radionuclides, or detachable molecule fragments having a typical mass, which can be detected in a mass spectrometer. Preferably, detachable molecule fragments have a single-positive or single-negative net charge for better detectability in the mass spectrometer. Preferably, the mass spectrometry detection is carried out and visualized using matrix assisted laser desorption/ionization mass spectrometry (MALDI), or using electron spray mass spectrometry (ESI).

The amplificates obtained are subsequently hybridized to an array or a set of oligonucleotides and/or PNA probes.

Preferably, where the amplificate nucleic acid is in solution, hybridization of the amplificates to the detection oligonucleotides or PNA oligomers is conducted in a hybridization chamber at a hybridization temperature that is dependant upon the selection of oligos. Optimal incubation temperatures and times will differ, depending on the particular oligonucleotides or PNA oligomers selected, and appropriate adjustments to the experimental setup can be readily determined by a person skilled in the art. Preferably, hybridization is carried out under moderately stringent to stringent conditions as defined herein above, or the art-recognized equivalent thereof. In a preferred embodiment, the hybridization is conducted at a temperature that is about 0.5° C. to 3° C. lower than the lowest melting temperature of the selected oligonucleotides, for 16 hours in an appropriate buffer solution. In a particular preferred embodiment, the buffer solution contains SSC and sodium laurel sarcosinate and the hybridizing temperature is 42° C. In a further embodiment the hybridization is conducted at a temperature of 45° C. for four hours. Preferably, the hybridization is carried out in Unihybridization solution (1:4 dilution v/v; Telechem).

Preferably, the set of probes used during the hybridization is comprises at least 10 oligonucleotides or PNA-oligomers. In the inventive method, the amplificates serve as probes which hybridize to oligonucleotides previously bonded to a solid phase. The non-hybridized fragments are subsequently removed.

Preferably, said oligonucleotides comprise at least one base sequence having a length of about 13 nucleotides, which is reverse complementary or identical to a segment of the amplificates sequences, wherein the segment comprises at least one CpG, TpG or CpA dinucleotide sequence. In a particularly preferred embodiment, said dinucleotide is located within the middle third of the oligonucleotide. The cytosine of the CpG dinucleotide is the 5^{th} to 9^{th} nucleotide from the 5′-end of the about 13-mer. Preferably, one oligonucleotide exists for each CpG dinucleotide of interest. More preferably, each CpG dinucleotide of interest is analyzed using two oligonucleotides, one comprising a CpG dinucleotide at the position in question and another comprising a TpG dinucleotide at the position in question.

More preferably, said oligonucleotides comprise at least one base sequence having a length of about 18 nucleotides, which is reverse complementary or identical to a segment of the amplificates sequences. Preferably the CpG dinucleotide is located between the 7^{th} and the 11^{th} nucleotide of said segment. Preferably, at least one CpG is located in the middle of said segment. Preferably, not more than two CpG dinucleotides are located in said segment.

Said oligonucleotides may also be in the form of peptide nucleic acids (PNA) comprising at least one base sequence having a length of about 9 bases which is reverse complementary or identical to a segment of the amplificates sequences, wherein the segment comprises at least one CpG dinucleotide. The cytosine of the CpG dinucleotide is the 4^{th} to 6^{th} nucleotide seen from the 5′-end of the about 9-mer. Preferably, one PNA oligomer exists for each CpG dinucleotide. More preferably, each CpG dinucleotide is analyzed by means of two PNA oligonucleotides, one comprising a CpG dinucleotide at the position in question and another comprising a TpG dinucleotide at the position in question.

Therefore, in a particularly preferred embodiments, two oligomers exist for each CpG position, one comprising a CpG dinucleotide at the dinucleotide position to be analysed, and the other comprising a TpG oligonucleotide at said position (i.e., one oligonucleotide specific for detection of methylated nucleic acids and the other specific for the detection of unmethylated versions of the same nucleic acid). The use of the two species of oligonucleotide on the solid phase enables an analysis of the degree of methylation within a genomic DNA sample. Comparison of the relative amount of nucleic acid hybridized to each species of oligonucleotide enables the deduction of the degree of methylation at the position in question.

In the final step of stage 1 of Step 4 of the method, the hybridized amplificates are detected. Preferably, labels attached to the amplificates are identifiable at each position of the solid phase at which an oligonucleotide sequence is located.

Preferably, the labels of the amplificates include, but are not limited to fluorescence labels, radionuclides, or detachable molecule fragments having a typical mass which can be detected in a mass spectrometer. Preferably, detection of the amplificates, detachable fragments of the amplificates or of probes which are complementary to the amplificates using mass spectrometry is by matrix assisted laser desorption/ionization mass spectrometry (MALDI) (e.g., Karas & Hillenkamp, Anal Chem., 60:2299-301, 1988), or using electron spray mass spectrometry (ESI). Preferably, the produced detachable mass fragments may have a single-positive or single-negative net charge for better detectability in the mass spectrometer.

Preferably, the array of different oligonucleotide- and/or PNA-oligomer sequences is arranged on the solid phase in the form of a rectangular or hexagonal lattice. The solid phase surface is preferably composed of silicon, glass, polystyrene, aluminum, steel, iron, copper, nickel, silver, or gold. However, nitrocellulose as well as plastics such as nylon which can exist in the form of pellets or also as resin matrices are possible as well.

Methods for manufacturing such arrays are well-known in the art, for example, from U.S. Pat. No. 5,744,3051 using solid-phase chemistry and photolabile protecting groups. An overview of the prior art in oligomer array manufacturing can be gathered from a special edition of Nature Genetics (Nature Genetics Supplement, Volume 21, January 1999), and from the literature cited therein.

Stage II of Step 4. The analysis of the methylation status of specific CpG positions within a number of samples generates a large amount of data. Sophisticated statistical and data-analysis techniques are applied to organize and analyze the data; that is, to correlate the methylation pattern with the phenotypic characteristics of the examined samples. Statistical analysis employing, for example, a T-test or a Wilcoxon test, can be used to determine the probability (‘p-value’) that the observed distribution of samples between the classes for each specific CpG position occurred by chance. Each CpG position is then ranked according to the p-values observed. Only the CpG positions of the appropriate p-value are used in the panel.

Once the panel is defined, algorithmic methods for the classification of a sample based on the methylation status of the CpG positions within the panel are developed. Preferably, the correlation of the methylation status of the marker CpG positions with the phenotypic parameters is done substantially without human intervention. Machine learning algorithms automatically analyse experimental data, discover systematic structure in it, and distinguish relevant parameters from uninformative ones.

Machine learning predictors are trained on the methylation patterns (CpG/TpG ratios) at the investigated CpG sites of the samples with known phenotypical classification. The CpG positions which prove to be discriminative for the machine learning predictor are used in the panel. In a particularly preferred embodiment of the method, both methods are combined; that is, the machine learning classifier is trained only on the CpG positions that are significantly differentially methylated according to the statistical analysis. This method is successful in cancer classification (Model, F., Adorjan, P., Olek, A., and Piepenbrock, C., Bioinformatics. 17 Suppl 1:157-164, 2001).

Thus, step 4 provides for comparing, among a plurality of test genomic DNA samples corresponding to different test tissues and/or subjects, and using, preferably, at least one of a medium- or a high-throughput controlled assay suitable therefore, the methylation states corresponding to the secondary differentially methylated CpG dinucleotide sequence, or to the pattern, whereby a reliable methylation marker is provided.

Step 5—Assay Design and panel Validation:

In a particularly preferred embodiment, the identified and selected CpG marker positions are further utilized in the design of an applied assay suitable for commercial clinical, diagnostic, research and/or high throughput application. Said applied assay may also be used to further validate the panel upon a larger sample set.

Several methods for the high throughput analysis of methylation within genomic DNA are available. These include restriction enzyme based analysis systems and more preferrably bisulphite based methodologies such as Ms SNuPE, hybridization analysis, MSP, and real time PCR based applications. Once a suitable diagnostic assay has been assembled, the gene panel is validated by analysis of a test run of samples numbering in their hundreds. A diagnostic assay is understood to have been validated if it performs to the required levels of sensitivity and specificity, typically this would be a minimum sensitivity of 75%, and a minimum specificity of 90%.

Preferred methods for use in a diagnostic and/or prognostic applied assays comprise bisulfite treatment of the genomic DNA, followed by a primer and/or probe based detection methodology.

Particularly preferred embodiements comprise the use of MSP, MS-SNuPE, oligonucleotide hybridization (as described in Step 4 herein), MethyLight™ or HeavyMethyl™ assays, or combinations thereof.

Fluorescence-based Real Time Quantitative PCR, and MethylLight™ assay. A particularly preferred embodiment comprises use of fluorescence-based Real Time Quantitative PCR (Heid et al., Genome Res. 6:986-994, 1996) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulphite treatment, the probe is preferably methylation specific, as described in U.S. Pat. No. 6,331,393, (hereby incorporated by reference) also known as the MethylLight™ assay. Variations on the TaqMan™ detection methodology that are also suitable for use with the described invention include the use of dual probe technology (Lightcycler™) or fluorescent amplification primers (Sunrise™ technology). Both these techniques may be adapted in a manner suitable for use with bisulphite treated DNA, and moreover for inventive methylation analysis of CpG dinucleotides.

HeavyMethy™. A further suitable method for assessment of methylation by analysis of bisulphite treated nucleic acids comprises the use of blocker oligonucleotides. The general use of such oligonucleotides has been described by Yu et al., BioTechniques 23:714-720, 1997. Blocking probe oligonucleotides are hybridized to the bisulphate-treated nucleic acid concurrently with the PCR primers. PCR amplification of the nucleic acid is terminated at the 5′ position of the blocking probe, thereby amplification of a nucleic acid is suppressed wherein the complementary sequence to the blocking probe is present. The probes may be designed to hybridize to the bisulphate-treated nucleic acid in a methylation status specific manner. For example, for detection of methylated nucleic acids within a population of unmethylated nucleic acids, suppression of the amplification of nucleic acids that are unmethylated at the position in question would be carried out by the use of blocking probes comprising a ‘CpG’ at the position in question, as opposed to a ‘CpA’ dinucleotide sequence, such as has been described in the German patent application DE 101 12 515.

MS-SNuP. In a further preferred embodiment, the determination of the methylation status of the CpG positions comprises use of template-directed oligonucleotide extension, such as “Ms-SNuPE” (Methylation-sensitive Single Nucleotide Primer Extension), described by Gonzalgo & Jones, Nucleic Acids Res. 25:2529-2531, 1997.

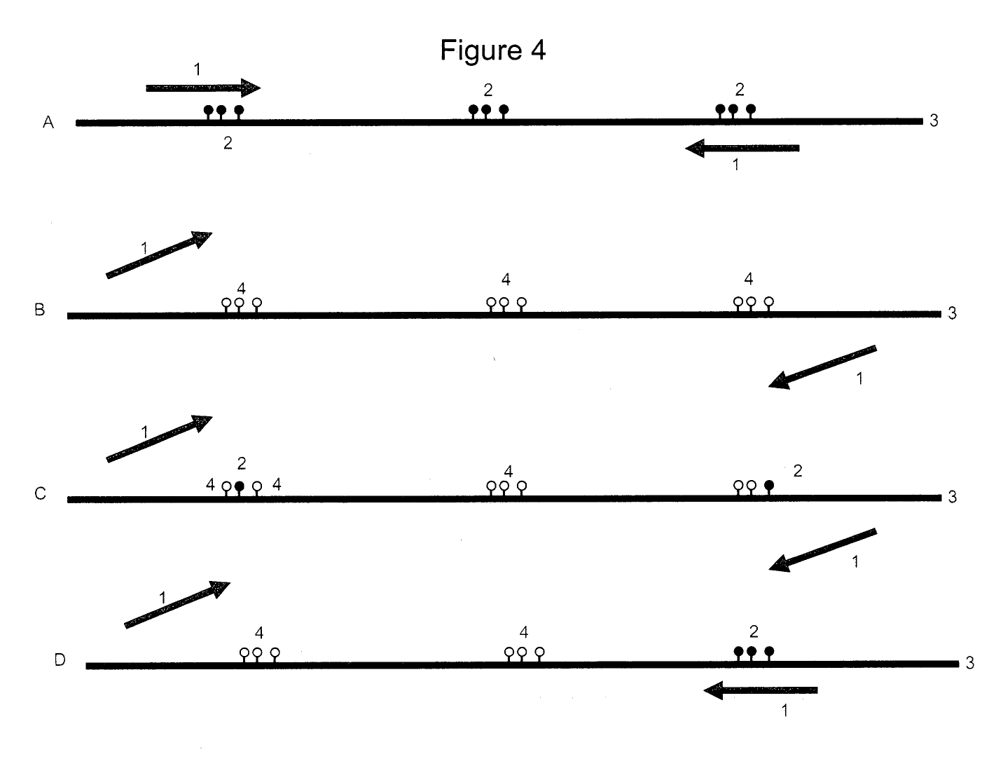

MSP. MSP (Methylation-specific PCR) refers to the art-recognized methylation assay described by Herman et al. Proc. Natl. Acad. Sci. USA 93:9821-9826, 1996, and by U.S. Pat. No. 5,786,146. In MSP applications, the use of methylation status specific primers for the amplification of bisulphate-treated DNA allows for distinguishing between methylated and unmethylated nucleic acids. MSP primer pairs contain at least one primer which hybridizes to a bisulphate-treated CpG dinucleotide of a pre-specified methylation state. Therefore, the sequence of said primers comprises at least one CpG, TpG or CpA dinucleotide. MSP primers specific for non-methylated DNA contain a ‘T’ at the 3′ position of the C-position in the CpG dinucleotide. Detection of the amplificate allows for the determination of the presence of a methylated nucleic acid. The use of MSP thereby allows for the detection of a nucleic acid of a pre-specified methylation state to be amplified against a background of alternatively methylated nucleic acids (see FIG. 4 herein and the accompanying description).

FIG. 4 shows the polymerase mediated amplification of a CpG-rich sequence using methylation specific primers on four representative bisulfite-treated DNA strands (example cases “A”-“D”) (“MSP Amplification”). The methylation specific forward and reverse primers (“1”), in each case, can anneal to the bisulfite-treated DNA strand (“3”) if the corresponding subject genomic CpG sequences were methylated. The bisulfite-treated DNA strand (“3”) can be amplified if both forward and reverse primers (“1”) anneal, as shown in representative case “A” at the top of the figure. The arrows (1) represent primers, and dark circular marker positions (2) on the DNA strand (3) represent methylated bisulfite-converted CpG positions, whereas white positions (4) represent unmethylated bisulfite-converted positions. The top example “A” strand, represents the case where all the subject genomic CpG positions were co-methylated, and both forward and reverse primers are thereby able to anneal with and amplify the corresponding treated nucleic acid. For the example “B” strand, none of the subject genomic CpG positions were methylated, therefore none of the primers anneal to the corresponding treated nucleic acid sequence and the sequence is not amplified. For example “C” strand, the three subject genomic CpG positions covered by the forward and reverse primers are not co-methylated (only one of said positions is methylated), and therefore, subsequent to bisulfite treatment of the DNA the primers do not anneal. For the fourth example “D” strand, the positions covered by the reverse primer were methylated CpG sequences in the subject genomic DNA, and the reverse primer thus anneals to the corresponding bisulfite-treated sequence. However, there is no exponential amplification of the corresponding bisulfite-treated DNA sequence, because the subject genomic CpG positions covered by the forward primer were not methylated and the forward primer does not anneal.

The use of each of these techniques is discussed in more detail in the following description of a preferred embodiment of the applied assay, comprising the following steps:

i) treating the DNA such that all umnethylated cytosine bases are converted to uracil and wherein 5-methylcytosine bases remain unconverted;

ii) amplifying of one or more of the CpG positions identified in 1.5) using at least 2 primer oligonucleotides;

iii) detecting the amplificate nucleic acids;

iv) determining the methylation state of said CpG positions; and

v) determining of one or more of the phenotypic parameters identified in 1.1)

In a particularly preferred embodiment, the treatment of step i) is carried out by means of chemical treatment, most preferably by means of treatment with a solution of bisulfite. It is preferred that the DNA is embedded in agarose before said treatment to keep the DNA in the single-stranded state during treatment, or, by treatment in the presence of a radical trap and a denaturing reagent, preferably an oligoethylene glycol dialkyl ether or, for example, dioxane. Prior to the PCR reaction, the reagents are removed either by washing in the case of the agarose method, or by standard art recognized DNA purification methods (e.g., precipitation or binding to a solid phase, membrane) or, simply by diluting in a concentration range that does not significantly influence the PCR.

Where the aim of the applied assay is the detection of at least one treated nucleic acid that was, prior to treating in step (i), of a predetermined methylation status (either methylated or unmethylated), said nucleic acids shall hereinafter be referred to as ‘target nucleic acids’ or ‘target DNA’. The nucleic acids present in the reaction that were, prior to said treatment, of the alternative methylation status shall hereinafter be referred to as ‘background DNA’ or ‘background nucleic acids.’ For example, wherein the aim of the method is the detection of methylated nucleic acids, in step (ii), treated nucleic acids that were unmethylated prior to such treatment are referred to as ‘background DNA,’ whereas treated nucleic acids that were prior to such treatment methylated are referred to as ‘target DNA’. In one preferred embodiment, the background DNA is present at 100 times the concentration of the target DNA. In a further preferred embodiment, the background DNA is present at 1000 times the concentration of the target DNA.

In a particular embodiment, only nucleic acids of a predetermined methylation status are amplified in step (ii); that is, EITHER positions that were methylated prior to treatment are preferentially amplified over positions that were unmethylated prior to treatment, OR positions that were unmethylated prior to treatment are preferentially amplified over positions that were methylated prior to treatment (i.e., target DNA is preferentially amplified over background DNA). In a preferred embodiment, this may be achieved by PCR amplification with added blocking oligonucleotides, or, in an alternative embodiment, by means of methylation specific primers.

In a particularly preferred embodiment, the applied assay further comprises the use of at least one probe oligonucleotide which hybridizes to said one or more marker CpG positions identified in the previous stages of the method (island discovery, marker validation, etc.). Said probe oligonucleotides preferentially hybridize either to positions that were methylated prior to bisulfite treatment or to positions that were unmethylated prior to bisulfite treatment (i.e., either to background DNA or to target DNA).

Variants of the applied assay may utilize one or more of the following species of probe oligonucleotides: blocking oligonucleotides, used during step ii) of the assay to afford preferential amplification of background over target DNA; hybridization oligonucleotides, as recited in the marker identification Step 4 of the method, used for hybridizing to the amplificate nucleic acid in step iii) of the assay to enable identification of the pre-treatment methylation status of selected CpG positions. In an alternative embodiment, the hybridization oligonucleotides are referred to as ‘reporter oligonucleotides,’ which are suitably labeled (e.g., dual labeled) for use in a real-time PCR-based analysis of the target DNA amplificate.

The use of the term ‘primer’ shall hereinafter be interpreted to mean an oligonucleotide that is used as a primer for the amplification of a nucleic acid.

In a particularly preferred embodiment of the general method and/or applied assay, at least one primer (e.g., blocking, hybridization, and/or reporter oligonucleotide) is at least 18-bases in length.

In one embodiment of the general method and/or applied assay, at least one primer (e.g., blocking, hybridization, and/or reporter oligonucleotide) comprises a 5′-CpG-3′ dinucleotide or a 5′-TpG-3′ dinucleotide or a 5′-CpA-3′-dinucleotide, thereby enabling the differentiation between target and background bisulphate-treated nucleic acids. It is further preferred that said dinucleotide is in the middle third of the oligonucleotide.

Blocking Oligonucleotides and Uses Thereof:

In one embodiment of the method, at least one, and preferably two or more blocking oligonucleotides are used in step ii) of the applied assay to allow for selective amplification of the target over background DNA.

The term ‘binding site’ refers herein to a sequence of the target nucleic acid and/or background nucleic acid that is reverse complementary to that of the oligonucleotides and/or primers and to which it therefore hybridizes.

In one embodiment of the method, the binding site of the at least one blocking oligonucleotide is identical to, or overlaps with that of the primer and thereby hinders the hybridization of the primer to its binding site.

In a particularly preferred embodiment of the method, the target DNA is DNA that was methylated prior to the treatment of step i) of the method of the assay, and background DNA, with respect to particular CpG sequences, is that which was unmethylated prior to step i) of the method. In this particularly preferred embodiment, the probe oligonucleotide is complementary to the treated sequence of the background DNA and thereby suppresses amplification of said background DNA and the treated target DNA is thereby preferentially amplified.

In a further preferred embodiment of the method, two or more such blocking oligonucleotides are used. In a particularly preferred embodiment, the hybridization of one of the blocking oligonucleotides hinders the hybridization of a forward primer, and the hybridization of another of the probe (blocker) oligonucleotides hinders the hybridization of a reverse primer that binds to the amplificate product of said forward primer.

In an alternative embodiment of the method, the blocking oligonucleotide hybridizes to a location between the reverse and forward primer positions of the treated background DNA, thereby hindering the elongation of the primer oligonucleotides.

It is particularly preferred that the blocking oligonucleotides are present in at least 5 times the concentration of the primers.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase. In a further preferred embodiment of the method, the fifth step of the method comprises the use of template-directed oligonucleotide extension, such as MS-SNuPE as described by Gonzalgo & Jones, Nucleic Acids Res. 25:2529-2531, 1997.

Preferably, several fragments are simultaneously enzymatically amplified in step (ii) of the applied assay, most preferably six or more fragments; that is, the assay preferably comprises a multiplex PCR analysis. Care must be taken in design of the assay to ensure that neither the primers, nor the probe oligonucleotides are complementary to one another, and thereby preclude formation of oligonucleotide dimers that hinder amplification of the treated DNA. Significantly, the design of the primer and probe oligonucleotides is aided by the fact that the two strands of a methylated bisulphate treated DNA have very different G/C contents. One strand is G-rich, the complement to that is C-rich. Therefore, a forward primer can never function also as a reverse primer which in turn ameliorates primer and probe design and facilitates the multiplexing.

It is particularly preferred that in step (iii) of the applied assay, the amplificate nucleic acids are detected. All possible known molecular biological methods may be used for this detection, including, but not limited to gel electrophoresis, sequencing, liquid chromatography, hybridizations, or combinations thereof. This step of the applied assay further acts as a qualitative control of the preceding steps.

In step (iv) of the applied assay, the methylation status of the marker CpG positions is determined by analysis of the amplificate nucleic acids(s). In one embodiment, multiple amplificate nucleic acids is analyzed by means of oligonucleotide hybridization analysis as described in method Step 4; most preferably using an arrayed format upon a solid phase.

In a further embodiment of the applied assay, step (iv) is carried out using MS-SNuPE analysis as described by Gonzalgo & Jones, Nucleic Acids Res. 25:2529-2531, 1997. It is particularly preferred that the Ms-SNuPE primer is at least fifteen but no more than twenty five nucleotides in length.

In a particularly preferred embodiment of the applied assay, steps (iii) and (iv) are carried out concurrently by use of reporter oligonucleotides or PNA oligomers. Said reporter oligonucleotide or PNA oligomer is identical to or reverse complementary to an at least 9-nucleotide long segment of the target sequence, wherein said reporter oligonucleotide comprises a 5′-CpG-3′ dinucleotide or a 5′-TpG-3′ dinucleotide or a 5′-CpA-3′-dinucleotide, thereby enabling the determination of the methylation status of one or more CpG positions (prior to the treatment of step (i) of the assay). The reporter oligonucleotide is detectably labeled and hybridizes to a binding site sequence of the amplificate nucleic acid thereby enabling the differentiation between target and background bisulphate-treated nucleic acids.

Said detectable labels may be any suitable labels used in the art (radioactive, mass labels, etc.), however it is particularly preferred that the labels are fluorescent dyes; thereby enabling the use of fluorescence-based detection technologies (e.g., fluorescence detection, fluorescence resonance energy transfer interactions, fluorescence polarization, etc.), wherein the presence of one or more target sequences is determined by means of an increase or decrease in fluorescence or fluorescence polarization.

An alternative embodiment of the method and/or applied assay further comprises the use of a fluorescent-labeled oligomer, which hybridizes directly adjacent to the reporter oligonucleotide and wherein said hybridization can be detected by means of fluorescence resonance energy transfer.

It is particularly preferred that the detection of the reporter oligonucleotide is carried out in a real-time manner by means of a TaqMan™ and/or LightCycler™ assay.

A particularly preferred variant of the method and/or applied assay comprises, in step (ii) of the assay, the use of at least one blocking oligonucleotide or PNA oligomer that hybridizes to a 5′-CpG-3′ dinucleotide or a 5′-TpG-3′ dinucleotide or a 5′-CpA-3′ dinucleotide, and thereby hinders the amplification of at least one background nucleic acid sequence, and wherein the detection carried out in step (iii) of the method is achieved by means of at least one reporter oligonucleotide that hybridizes to the amplificate of the target sequence, and thereby indicates the amplification of one or more target sequences.

In step (v) of the applied assay the methylation status of the marker CpG positions is correlated to phenotypic parameters of the individual (sample); that is, from the results of step (iv), a conclusion is reached as to which class (specified by its phenotypic parameters) the source of the analyzed DNA belongs to. This is carried out by means of the learning algorithm trained in Step 4 of the method, as described in detail herein above.

The ‘trained’ learning algorithm is applied to the methylation patterns of the sample to identify a sample as belonging to a specific class. In a preferred embodiment of the method and/or applied assay, said machine learning algorithm is a linear classifier (e.g., Support Vector Machines (SVM), perceptrons and Bayes Point Machines).

In a particular embodiment, the invention provides a kit comprising a bisulfite (or disulfite, or hydrogen sulfite) reagent, as well as oligonucleotides and/or PNA-oligomers suitable for use in an assay as described above.

In one embodiment of the invention, the described method and/or applied assay is used for the diagnosis of unwanted side-effects of: medicaments, cell proliferative disorders; dysfunctions, damages or diseases of the central nervous system (CNS); aggressive symptoms or behavioural disorders; clinical, psychological and social consequences of brain injuries; psychotic disorders and disorders of the personality; dementia and/or associated syndromes; cardiovascular diseases; malfunctions or damages, diseases, malfunctions or damages of the gastrointestine; diseases, malfunctions or damages of the respiratory system; injury, inflammation, infection, immunity and/or reconvalescence, diseases; malfunctions or damages as consequences of modifications in the developmental process; diseases, malfunctions or damages of the skin, muscles, connective tissue or bones; endocrine or metabolic diseases, malfunctions or damages, headache; and sexual malfunctions, or combinations thereof.

Particularly preferred is the use of the method and/or applied assay for the diagnosis of leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

In a particularly preferred embodiment, the described method and/or applied assay is used for the characterisation, classification, differentiation, grading, staging, and/or diagnosis of cell proliferative disorders, or the predisposition to cell proliferative disorders.

A further aspect of the invention provides a method for the treatment of a disease or medical condition which comprises a) diagnosing the disease phenotype of the patient according to the method or assay as described above; and b) providing a suitable treatment means for said diagnosed condition. In one embodiment, this method is used for the treatment of: medicaments, cell proliferative disorders; dysfunctions, damages or diseases of the central nervoussystem (CNS); aggressive symptoms or behavioural disorders; clinical, psychological and social consequences of brain injuries; psychotic disorders and disorders of the personality; dementia and/or associated syndromes; cardiovascular diseases; malfunctions or damages, diseases, malfunctions or damages of the gastrointestine; diseases, malfunctions or damages of the respiratory system; injury, inflammation, infection, immunity and/or reconvalescence, diseases; malfunctions or damages as consequences of modifications in the developmental process; diseases, malfunctions or damages of the skin, muscles, connective tissue or bones; endocrine or metabolic diseases, malfunctions or damages, headache; and sexual malfunctions, or combinations thereof.

Particularly preferred is the use of the method and/or applied assay for the treatment of leukemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile X syndrome, and Huntington's disease.

While the present invention has been described with specificity in accordance with certain of its preferred embodiments, the following example serves only to illustrate the invention and is not intended to limit the invention within the principles and scope of the broadest interpretations and equivalent configurations thereof. As used in this specification and the appended claims, the singular forms “a,” “an” and “the” include plural referents unless the content clearly dictates otherwise.

### EXAMPLE 1

### Identification of Novel and Reliable CpG Markers for the Diagnosis, Prognosis, and/or Staging of Colon Carcinoma

Step 1—Formulating a diagnostic aim for a methylation marker, and obtaining phenotypically distinguishable classes of biological samples comprising genomic DNA.

Formulation of diagnostic aim. The formulated diagnostic aim was identification of novel and reliable CpG methylation markers for the improved diagnosis and staging of colon carcinomas, wherein the defined phenotypic parameter was a presence or absence of a colon cell proliferative disorder selected from the group consisting of adenoma, metastatic carcinoma, non-metastatic carcinoma, and combinations thereof.

Obtaining phenotypically distinguishable classes of biological samples. Tissue samples were collected corresponding to the following stage classes of colon carcinoma: adenoma, metastatic carcinoma, non-metastatic carcinoma. Each tissue stage class was further segregated into sets of tissue stage classes according to additional variables; namely, according to different anatomical regions of the colon: ascending, descending, cecum, and sigmoid colon.

Additionally, corresponding normal samples were collected to enable comparison of the sets of disease stage classes with age-matched normal classes of adjacent tissues, and with normal peripheral blood lymphocytes.

Step 2—Identifying one or more primary differentially methylated CpG dinucleotide sequences using a controlled assay suitable for identifying at least one differentially methylated CpG dinucleotide sequences within the entire genome, or a representative fraction thereof.

All processes were performed on both pooled and/or individual samples, and analysis was carried out using two different Discovery methods; namely, methylated CpG amplification (MCA), and arbitrarily-primed PCR (AP-PCR).

AP-PCR. AP-PCR analysis was performed on sample classes of genomic DNA as follows:

1. DNA isolation; genomic DNA was isolated from sample classes using the commercially available Wizzard™ kit;

2. Restriction enzyme digestion; each DNA sample was digested with 3 different sets of restriction enzymes for 16 hours at 37° C.: RsaI (recognition site: GTAC); RsaI (recognition site: GTAC) plus HpaII (recognition site: CCGG; sensitive to methylation); and RsaI (recognition site: GTAC) plus MspI (recognition site: CCGG; insensitive to methylation);

3. AP-PCR analysis; each of the restriction digested DNA samples was amplified with the primer sets (SEQ ID NOS: 17-40) according to TABLE 1 at a 40° C. annealing temperature, and with ³²P dATP.

4. Polyacrylamide Gel Electrophoresis; 1.6 μl of each AP-PCR sample was loaded on a 5% Polyacrylamide sequencing-size gel, and electrophoresed for 4 hours at 130 Watts, prior to transfer of the gel to chromatography paper, covering the transferred gel with saran wrap, and drying in a gel dryer for a period of about 1-hour;

5. Autoradiographic Film Exposure; film was exposed to dried gels for 20 hours at −80° C., and then developed. Glogos was added to the dried gel and exposure was repeated with new film. The first autorad was retained for records, while the second was used for excising bands; and

6. Bands corresponding to differential methylation were visually identified on the gel. Such bands were excised and the DNA therein was isolated and cloned using the Invitrogen TA Cloning Kit.

TABLE 2 shows a selection of the AP-PCR results.

Selected cloned amplicons were sequenced in Step 3 of the method (see below).

TABLE 1

Primers of AP-PCR according to EXAMPLE 1, Step 2

| | | |
|---|---|---|
| PRIMER | SEQUENCE | SEQ ID NO: |

| | | |
|---|---|---|
| GC1 | GGGCCGCGGC | 17 |
| GC2 | CCCCGCGGGG | 18 |
| GC3 | CGCGGGGGCG | 19 |
| GC4 | GCGCGCCGCG | 20 |
| GC5 | GCGGGGCGGC | 21 |
| G1 | GCGCCGACGT | 22 |
| G2 | CGGGACGCGA | 23 |
| G3 | CCGCGATCGC | 24 |
| G4 | TGGCCGCCGA | 25 |
| G5 | TGCGACGCCG | 26 |
| G6 | ATCCCGCCCG | 27 |
| G7 | GCGCATGCGG | 28 |
| G8 | GCGACGTGCG | 29 |
| G9 | GCCGCGNGNG | 30 |
| G10 | GCCCGCGNNG | 31 |
| APBS1 | AGCGGCCGCG | 32 |
| APBS5 | CTCCCACGCG | 33 |
| APBS7 | GAGGTGCGCG | 34 |
| APBS10 | AGGGGACGCG | 35 |
| APBS11 | GAGAGGCGCG | 36 |
| APBS12 | GCCCCCGCGA | 37 |
| APBS13 | CGGGGCGCGA | 38 |
| APBS17 | GGGGACGCGA | 39 |
| APBS18 | ACCCCACCCG | 40 |

TABLE 2

Results of AP-PCR according to EXAMPLE 1, Step 2.

| | | | | | | | | |
|---|---|---|---|---|---|---|---|---|
| | Primer | Primer | Primer | | Tissue | methylation | Tissue | methylation |
| Experiment | 1 | 2 | 3 | band | Type 1 | state 1 | Type 2 | state 2 |

| | | | | | | | | |
|---|---|---|---|---|---|---|---|---|
| colon 4.1 | GC1 | G2 | APBS1 | 1 | colon nat | hypo | colon | hyper |
| | | | | | pool a1 | | pool a1 | |
| colon 4.1 | GC4 | G5 | APBS1 | 1 | colon nat | hypo | colon | hyper |
| | | | | | pool a1 | | pool a1 | |
| colon 4.2 | GC3 | G6 | APBS7 | 1 | colon nat | hypo | colon | hyper |
| | | | | | pool a1 | | pool a1 | |
| colon 4.2 | GC3 | G6 | APBS7 | 2 | colon nat | hypo | colon | hyper |
| | | | | | pool a1 | | pool a1 | |
| colon 4.2 | GC4 | G5 | APBS7 | 1 | colon nat | hypo | colon | hyper |
| | | | | | pool a1 | | pool a1 | |
| colon 4.2 | GC3 | G1 | APBS10 | 1 | colon nat | hypo | colon | hyper |
| | | | | | pool a1 | | pool a1 | |
| colon 4.2 | GC3 | G1 | APBS10 | 2 | colon nat | hypo | colon | hyper |
| | | | | | pool a1 | | pool a1 | |
| colon 4.2 | GC4 | G2 | APBS10 | 1 | colon nat | hyper | colon | hypo |
| | | | | | pool a1 | | pool a1 | |
| colon 4.5 | GC3 | G5 | APBS13 | 1 | colon nat | hypo | colon | hyper |
| | | | | | pool a1 | | pool a1 | |
| colon 4.5 | G3 | G4 | APBS17 | 1 | colon nat | hypo | colon | hyper |
| | | | | | pool a1 | | pool a1 | |
| colon 4.5 | G5 | G6 | APBS17 | 1 | colon nat | hypo | colon | hyper |
| | | | | | pool a1 | | pool a1 | |
| colon 4.6 | G7 | G8 | APBS13 | 1 | colon nat | hypo | colon | hyper |
| | | | | | pool a1 | | pool a1 | |
| colon 4.6 | G8 | G10 | APBS13 | 1 | colon nat | hypo | colon | hyper |
| | | | | | pool a1 | | pool a1 | |
| colon 4.6 | G5 | G7 | APBS12 | 1 | colon nat | hypo | colon | hyper |
| | | | | | pool a1 | | pool a1 | |
| colon 4.7 | G2 | G4 | APBS12 | 1 | colon nat | hypo | colon | hyper |
| | | | | | pool a1 | | pool a1 | |
| colon 4.7 | G1 | G3 | APBS11 | 1 | colon nat | hypo | colon | hyper |
| | | | | | pool a1 | | pool a1 | |
| colon 4.7 | G1 | G3 | APBS11 | 2 | colon nat | hypo | colon | hyper |
| | | | | | pool a1 | | pool a1 | |
| colon 4.8 | G1 | G8 | APBS10 | 1 | colon nat | hypo | colon | hyper |
| | | | | | pool a1 | | pool a1 | |
| colon 4.8 | G5 | G9 | APBS7 | 1 | colon nat | hyper | colon | hypo |
| | | | | | pool a1 | | pool a1 | |
| colon 4.8 | G2 | G6 | APBS5 | 1 | colon nat | hypo | colon | hyper |
| | | | | | pool a1 | | pool a1 | |
| colon 4.8 | G1 | G5 | APBS5 | 1 | colon nat | hypo | colon | hyper |
| | | | | | pool a1 | | pool a1 | |
| colon 4.8 | G4 | G10 | APBS5 | 1 | colon nat | hypo | colon | hyper |
| | | | | | pool a1 | | pool a1 | |
| colon 4.9 | G1 | G7 | APBS1 | 1 | colon nat | hypo | colon | hyper |
| | | | | | pool a1 | | pool a1 | |
| colon 4.9 | APBS10 | APBS13 | SPBS17 | 1 | colon nat | hypo | colon | hyper |
| | | | | | pool a1 | | pool a1 | |

MCA. MCA was used to identify hypermethylated sequences in one population of genomic DNA as compared to a second population by selectively eliminating sequences that do not contain the hypermethylated regions. This was accomplished, as described in detail herein above, by digestion of genomic DNA with a methylation-sensitive enzyme that cleaves un-methylated restriction sites to leave blunt ends, followed by cleavage with an isoschizomer that is methylation insensitive and leaves sticky ends. This is followed by ligation of adaptors, amplicon generation and subtractive hybridization of the tester population with the driver population.

In the initial restriction digestion reactions, 5 μg of each genomic DNA pool was digested with SmaI in a 100 μL reaction overnight at 25° C. in NEB buffer 4+BSA, and 100 units of enzyme (10 μL). The pools were then further digested with Xma I (2 μL=100 U), 6 hours at 37° C.

500 ng of the cleaned-up, digested material was ligated to the adapter-primer RXMA24+RXMA12 (Sequence: RXMA24: AGCACTCTCCAGCCTCTCACCGAC (SEQ ID NO:1); RXMA12: CCGGGTCGGTGA (SEQ ID NO:2). These were hybridized to create the adapter by heating together at 70° C. and slowly cooling to room temperature (RT) in a 30 μL reaction overnight at 16° C., with 400 U (1 μL) of T4 ligase enzyme.

3 μL of the ligation mix for both tester and driver populations was used in each initial PCR to generate the starting amplicons. Two PCR reactions were run for the tester, and 8 for the driver. Reactions were 100 μL, with 1 μL of 100 μM primer RXMA24 (SEQ ID NO: 1), 10 μL PCR buffer, 1.2 μL 25 mM dNTPs, 68.8 μl water, 1 μL titanium Taq, 2 μL DMSO, and 10 μL 5M Betaine. PCR comprised an initial step at 95° C. for 1 minute, followed by 25 cycles at 95° C. for 1 minute, followed by 72° C. for 3 minutes, and a final extension at 72° C. for 10 minutes.

The tester amplicons were then digested with XmaI as described above, yielding overhanging ends, and the driver amplicons were digested with SmaI as above, yielding blunt end fragments.

A new set of adapter primers (hybridized as described for the above RXMA primers) JXMA24+JXMA12 (Sequence: JXMA24: ACCGACGTCGACTATCCATGAACC (SEQ ID NO:3); JXMA12: CCGGGGTTCATG (SEQ ID NO:4)) was ligated to the Tester only (using the same conditions as described above for the RXMA primers).

Five μg of digested tester and 40 μg of digested driver amplicons were hybridized in a solution containing 4 μL EE (30 mM EPPS, 3 mM EDTA) and 1 μL of 5 M NaCl at 67° C. for 20 hours. A selective PCR reaction was done using primer JXMA24 (SEQ ID NO:3). The PCR amplification steps were as follows: an initial fill-in step at 72° C. for 5 minutes, followed by 95° C. for 1 minute, and 72° C. for 3 minutes, for 10 cycles. Subsequently, 10 μL of Mung Bean nuclease buffer plus 10 μL Mung Bean Nuclease (10U) was added and incubated at 30° C. for 30 minutes. This reaction was cleaned up and used as a template for 25 more cycles of PCR using JXMA24 primer and the same conditions.

The resulting PCR product (tester) was digested again using XmaI, as described above, and a third adapter, NXMA24 (AGGCAACTGTGCTATCCGAGTGAC; SEQ ID NO:5)+NXMA12 (CCGGGTCACTCG; SEQ ID NO:6) was ligated. The tester (500 ng) was hybridized a second time to the original digested driver (40 μg) in 4 μL EE (30 mM EPPS, 3 mM EDTA) and 1 μL 5 M NaCl at 67° C. for 20 hours. Selective PCR was performed using NXMA24 primer (SEQ ID NO:5) as follows: an initial fill-in step at 72° C. for 5 minutes, followed by 95° C. for 1 minute, and 72° C. for 3 minutes, for 10 cycles. Subsequently, 10 μL of Mung Bean nuclease buffer plus 10 mL Mung Bean Nuclease (10U) was added and incubated at 30° C. for 30 minutes. This reaction was cleaned up and used as a template for 25 more cycles of PCR using NXMA24 primer and the same conditions.

The resulting PCR product (1.8 μg) was digested with XmaI (in 50 μL total volume, NEB buffer 4+BSA, and 2 μL=100 U XmaI, 6 hours at 37° C.) and ligated into the vector pBC Sk—predigested with XmaI and phosphatased (675 ng). 5 μL of a 30 μL ligation was used to transform chemically competent TOP10™ cells according to the manufacturer's instructions. The transformations were plated onto LB/XGaI/IPTG/CAM plates. Selected insert colonies were sequenced in Step 3 of the method.

Scoring. All identified MeSTs were scored according to the following criteria (each parameter scoring one point, positive or negative as indicated): location in the genome within a CpG island (positive); near a predicted or known gene (positive); part of a repetitive element of the genome (negative); location in reference to a gene promoter region (positive); coding region (positive); intron (positive); 3′ region (positive); location in reference to a gene known to be associated with cancer (e.g., the gene is a member of a class associated with cancer development, such as transcription factor, growth factor, etc.) (positive); presence in more than one pool of the experiment (positive).

A summary of the MeST positions as scored in Step 2 can be seen in TABLE 3.

TABLE 3
Stage 3 Scored MeSTs

| | | | | | # of | # of |
|---|---|---|---|---|---|---|
| | | | | | Amplicons/ | oligos/ |
| EpiID | Score | METHOD | COMPARISON | GENE | fragment | amplicon |
| 15628 | 1 | Appcr | Colon cancer vs normal | RING FINGER PROTEIN | 1 | 16 |
| 15660 | 4 | Appcr | Colon cancer vs normal | HOMEOBOX PROTEIN | 2 | 20 |
| 15805 | 3 | Appcr | Colon cancer vs normal | HOMEOBOX PROTEIN | 2 | 6 |
| 15799 | 3 | Appcr | Colon cancer vs normal | Transcription factor | 2 | 12 |
| 15872 | 2 | Appcr | Colon cancer vs normal | No gene | 2 | 22 |
| 15694 | 1 | MCA | Colon vs PBLs | Unknown gene- | 1 | 4 |
| | | | | hypermethylated in PBL's vs | | |
| | | | | colon | | |
| 15693 | 2 | MCA | Colon vs PBLs | HOMEOBOX PROTEIN; | 1 | 2 |
| | | | | colon vs PBL | | |
| 15862 | 2 | Appcr | Colon vs PBLs | PROTEIN (FRAGMENT) | 1 | 2 |
| | | | | colon vs PBL | | |
| 15873 | 1 | Appcr | Colon cancer vs normal | No gene-2 exp | 1 | 2 |
| 15665 | 4 | Appcr | Colon cancer vs normal | Transcription factor | 2 | 8 |
| 15798 | 1 | Appcr | Colon cancer vs normal | AMINO ACID transporter | 2 | 14 |
| 15810 | 2 | Appcr | Colon cancer vs normal | No gene within island | 2 | 14 |
| 15782 | 3 | MCA | Colon cancer vs normal | Cadherin-like | 1 | 20 |
| 15839 | 2 | Appcr | Colon cancer vs normal | No gene within island | 2 | 6 |
| 15752 | 2 | MCA | Colon cancer vs normal | 5 azacytidine induced | 2 | 8 |
| 15714 | 4 | Appcr | Colon cancer vs normal | TUMOR NECROSIS FACTOR | 1 | 8 |
| | | | | RECEPTOR SUPERFAMILY | | |
| | | | | MEMBER | | |
| 15667 | 4 | Appcr | Colon cancer vs normal | TRANSMEMBRANE | 2 | 6 |
| | | | | PROTEIN | | |
| 15724 | 1 | MCA | Colon cancer vs normal | PROTEIN | 2 | 6 |
| 15701 | 2 | Appcr | Colon cancer vs normal | adenylate cyclase | 2 | 6 |
| 15896 | 1 | Appcr | Colon cancer vs normal | No gene | 1 | 6 |
| 15747 | 0 | MCA | Colon cancer vs normal | Hypothetical protein-leucine | 3 | 18 |
| | | | | rich repeat | | |
| 15868 | 2 | Appcr | Colon cancer vs normal | TRANSCRIPTION | 2 | 18 |
| | | | | INITIATION FACTOR | | |
| 15792 | 3 | Appcr | Colon cancer vs normal | PROBABLE G PROTEIN- | 1 | 8 |
| | | | | COUPLED RECEPTOR | | |
| 15814 | 3 | Appcr | Colon cancer vs normal | COREPRESSOR | 1 | 2 |
| 15695 | 3 | MCA | Colon cancer vs normal | Transforming Growth Factor | 1 | 6 |
| | | | | Beta Binding Protein | | |
| 15789 | 3 | Appcr | Colon cancer vs normal | HOMEOBOX PROTEIN | 1 | 4 |
| 15804 | 4 | Appcr | Colon cancer vs normal | Transcription factor | 1 | 2 |
| 15812 | 0 | Appcr | Colon cancer vs normal | No gene | 1 | 4 |
| 15830 | 4 | Appcr | Colon cancer vs normal | HOMEOBOX PROTEIN | 1 | 16 |
| 15850 | 1 | Appcr | Colon cancer vs normal | Homo sapiens mRNA for | 1 | 4 |
| | | | | KIAA protein | | |
| 15672 | 6 | Appcr | Colon cancer vs normal | Cancer asssociated protein | 1 | 6 |
| 15712 | 5 | Appcr | Colon cancer vs normal | RING FINGER PROTEIN | 2 | 12 |
| 2385 | | LIT | | Transcription factor | 1 | 14 |
| 2064 | | RP1 | | Oncogene | 1 | 2 |
| 2383 | | RP1 | | Extracellular matrix protein | 1 | 2 |
| 2393 | | RP1 | | TRANSMEMBRANE | 1 | 2 |
| | | | | PROTEIN | | |
| 2322 | | RP1 | | Tumor protein | 1 | 20 |
| 2044 | | RP1 | | Proteoglycan | 1 | 6 |
| 2037 | | RP1 | | Antigen | 1 | 18 |
| 2004 | | RP1 | | Tumor suppressor | 1 | 10 |
| 2188 | | RP1 | | Candidate tumor suppressor | 1 | 8 |
| 2267 | | RP1 | | growth factor receptor | 1 | 2 |
| 2382 | | RP1 | | Extracellular matrix protein | 1 | 8 |
| 401 | | RP1 | | Antigen | 1 | 22 |
| 2056 | | Control-X | | oncogene family | 1 | 4 |

Thus, step 2 provides for identifying one or more primary differentially methylated CpG dinucleotide sequences of a test subject genomic DNA using a controlled assay suitable for identifying at least one differentially methylated CpG dinucleotide sequences within the entire genome, or a representative fraction thereof.

Step 3 —Determination of the characteristic methylation patterns of CpG positions in the vicinity of the differentially methylated CpG positions identified in Step 2 above, and thereby determining further CpG positions differentially methylated between the sample classes.

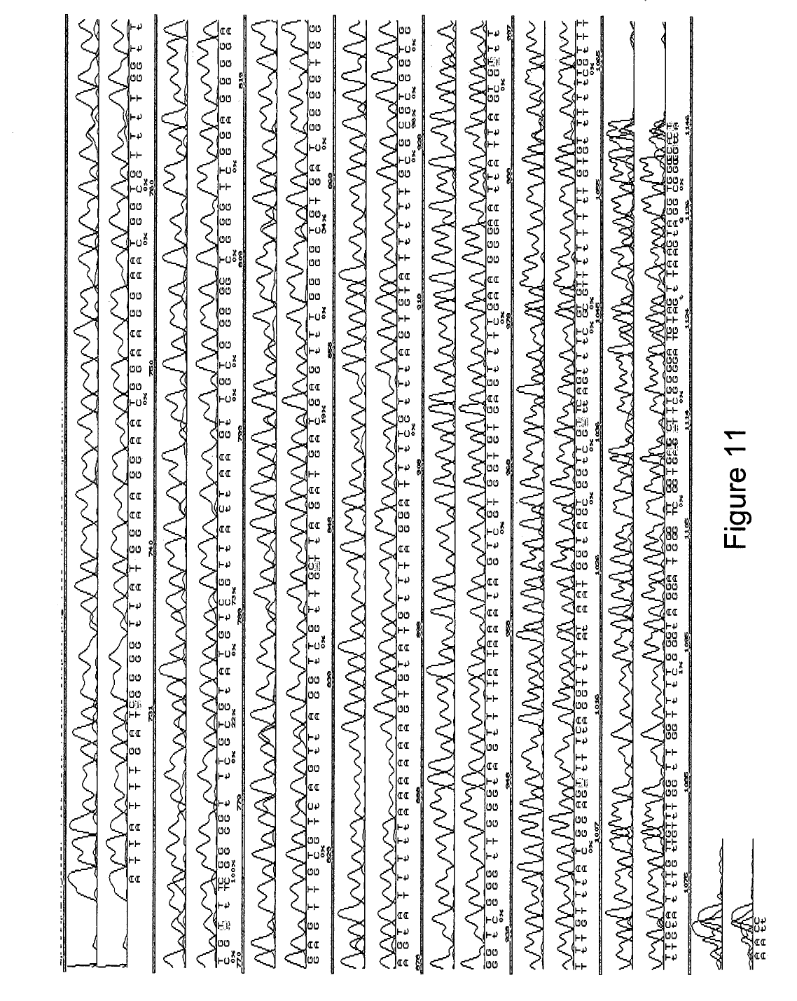

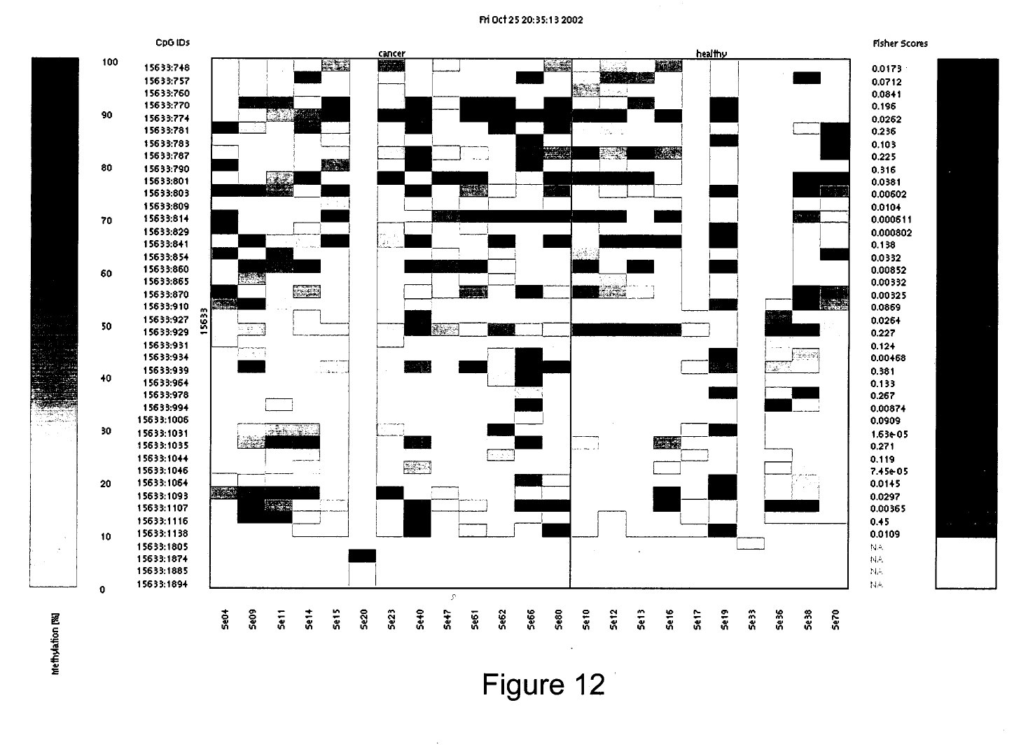

All identified MeSTs were further investigated by means of DNA sequencing. The genomic DNA of interest was bisulfite-treated and sequenced. The sequencing output was then processed using proprietary software, the output of which can be seen in FIGS. 11 and 12.

FIG. 11 shows the sequence analysis of MeST number 15633, by sequencing of the pooled colon carcinoma samples. The upper trace of each trace pair shows the sequencing output prior to processing, the lower trace shows the trace post-processing. At each CpG dinucleotide, the relative amount of methylation present in the sample was determined, as can be seen from the trace only three positions were found to be significantly methylated (position 775 at 100%; position 790 at 73%, and position 929 at 96%).

FIG. 12 shows the sequencing analysis of specific CpG positions of MeST number 15633, within individual samples. Each horizontal line represents a specific CpG site. Each vertical column represents a different sample. Blue-colored boxes represent a methylated status, and yellow-colored boxes represent an unmethylated status. An intermediate status is represented by a shades of green, according to the color bar at the left of the Figure. Failures are represented by white fields.

The sequence was not determined to have a sufficiently high CpG density to provide a utilitarian basis for assay design. This sequence was therefore not analysed in the further steps of the method.

Thus, step 3 provides for identifying, within a genomic DNA ‘context’ region surrounding or including one or more primary differentially methylated CpG dinucleotides, and using an assay suitable therefore, one or more secondary differentially methylated CpG dinucleotide sequences, or a pattern having a plurality of differentially methylated CpG dinucleotide sequences and including the primary and at least one secondary differentially methylated CpG dinucleotide sequences.

Step 4—Analyzing the methylation status of differentially methylated CpG positions identified in Step 3 within larger numbers of biological samples of each class of interest to identify CpG positions suitable for reliably distinguishing between or among classes of DNA either alone or in combination with other CpG positions.

The following is a gene methylation analysis used to compare the methylation states of colon adenoma and colon carcinoma sample classes. Multiplex PCR was carried out upon tissue sample classes originating from colon adenomas or colon carcinomas. Multiplex PCR was also carried out upon corresponding healthy colon tissue samples.

In stage I of this step, each sample was treated with a bisulfite solution and subjected to multiplex PCT analysis to deduce the methylation status of CpG positions.

In stage II of this step, the CpG methylation information for each sample was collated and used in a comparative data analysis.

Stage I. In the first stage, the genomic DNA was isolated from the cell samples using the Wizzard™ kit from (Promega).

The isolated genomic DNA from the samples was treated using a bisulfite solution (e.g., hydrogen sulfite, or disulfite), such that all non-methylated cytosines within the sample are converted to thymidine, whereas all 5-methylated cytosines within the sample remain unmodified.

The treated nucleic acids were amplified using multiplex PCR reactions, amplifying 8 fragments per reaction with Cy5 fluorescently-labeled primers. The multiplex PCR solution and cycle conditions were as follows:

Reaction solution: 10 ng bisulfite-treated DNA; 3.5 mM MgCl2, 400 μM dNTPs; 2 pmol each primer; 1 U Hot Star Taq (Qiagen); and

Cycle conditions; forty cycles were carried out as follows: denaturation at 95° C. for 15 min, followed by annealing at 55° C. for 45 sec., primer elongation at 65° C. for 2 min. Additionally, a final elongation at 65° C. was carried out for 10 min.

All PCR products from each individual sample were then hybridized to glass slides carrying a pair of immobilized oligonucleotides for each CpG position under analysis. Each of these immobilized detection oligonucleotides was designed to hybridize to a bisulphite-converted binding site corresponding to the sequence around a particular genomic CpG sequence that was either originally unmethylated (and thus converted by bisulfite to UgG, and then to TpG during amplification) or methylated (and thus remaining as CpG during amplification). Hybridization conditions were selected (e.g., moderately stringent to stringet) to allow the detection of the single nucleotide differences between the post bilsulfite TpG and CpG variants.

A 5 μl volume of each multiplex PCR product was diluted in 10×Ssarc buffer (10×Ssarc comprises: 230 ml of 20×SSC; 180 ml of 20% sodium lauroyl sarcosinate solution; and distilled H₂O to 1000 ml). The reaction mixture was then hybridized to the detection oligonucleotides as follows: denaturation at 95° C.; cooling to 10° C.; and hybridization at 42° C. overnight, followed by washing with 10×Ssarc and dH₂O at 42° C.

Fluorescent signals from each hybridized oligonucleotide were detected using Genepix™ scanner and software. Ratios, for each CpG position, for the two signals (i.e., between the CpG oligonucleotide- and the TpG oligonucleotide-related signals) were calculated, based on comparison of intensity of the fluorescent signals.

Stage II. The data obtained according to stage I was sorted into a ranked matrix according to CpG methylation differences between or among the two classes of tissues, using an algorithm.

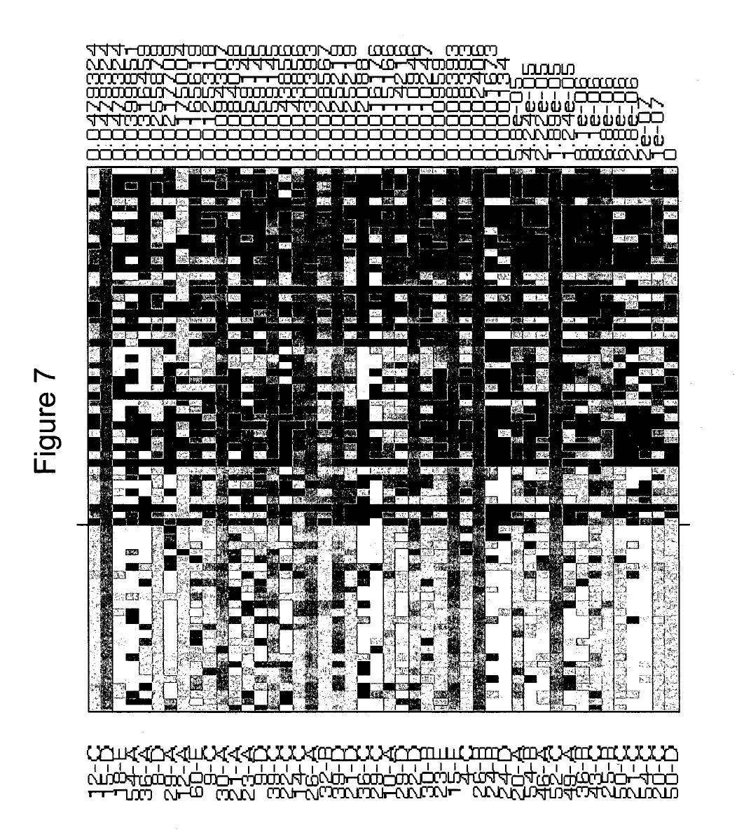

FIGS. 7 to 10 show a sub-selection of this ranked data. The most significant CpG positions are at the bottom of the matrix with significance decreasing towards the top. Black indicates total methylation at a given CpG position, white represents' no methylation at the particular position, with degrees of methylation represented in gray, from light (low proportion of methylation) to dark (high proportion of methylation).

With respect to each of FIGS. 7 to 10, each row represents one specific CpG position within a gene, and each column shows the methylation profile for the corresponing CpG positions for different samples within the two sample classes being compared. Both CpG position and gene identifiers are shown on the left side of the FIGS. 7-10, and these indices are cross-referenced with TABLE 4 below to identify the gene in question and thus the particular detection oligomer used. Additionally, p-values for the individual CpG positions are shown on the right side of these FIGS. 7 to 10. The p-values are the probabilities that the observed distribution occurred by chance in the data set.

For selected distinctions, we trained a learning algorithm (support vector machine, SVM™). The SVM (as discussed by F. Model, P. Adorjan, A. Olek, C. Piepenbrock, 17 Suppl 1:S157-64, 2001) constructs an optimal discriminant between two classes of given training samples. In this case, each sample is described by the methylation patterns (CpG/TpG ratios) at the investigated CpG sites. The SVM was trained on a subset of samples of each class, which were presented with the diagnosis attached. Independent test samples, which were not previously shown to the SVM, were then presented to evaluate whether the diagnosis can be predicted correctly based on the predictor created in the training round. This procedure was repeated several times using different partitions of the samples, a method called cross-validation. Significantly, all rounds are performed without using any knowledge obtained in the previous runs. The number of correct classifications was averaged over all runs, which gives a good estimate of our test accuracy (percent of correct classified samples over all rounds).

TABLE 4
Index of numerical gene identifiers and gene names
corresponding to FIGS. 7-10.

| | |
|---|---|
| NUMBER IN FIGURES | GENE NAME |

Healthy vs Non-Healthy

| | |
|---|---|
| 50-D | CDH13 |
| 20-C | CD44 |
| 54-C | TPEF (=TMEFF2; =HPP1) |
| 21-C | CSPG2 |
| 50-C | CDH13 |
| 25-B | GSTP1 |
| 43-C | TGFBR2 |
| 36-B | N33 |
| 49-A | CAV1 |
| 52-C | PTGS2 |
| 46-A | TP73 |
| 54-B | TPEF (=TMEFF2; =HPP1) |
| 20-A | CD44 |
| 24-D | ERBB2 |
| 24-B | ERBB2 |
| 26-B | GTBP/MSH6 |
| 4-C | EGR4 |
| 15-E | CDH1 |
| 23-E | EGFR |
| 30-B | LKB1 |
| 22-D | DAPK1 |
| 29-D | IGF2 |
| 10-A | HLA-F |
| 29-C | IGF2 |
| 36-C | N33 |
| 21-D | CSPG2 |
| 39-D | PTEN |
| 32-B | MLH1 |
| 26-A | GTBP/MSH6 |
| 14-C | CALCA |
| 22-C | DAPK1 |
| 39-C | PTEN |
| 9-D | WT1 |
| 23-A | EGFR |
| 21-A | CSPG2 |
| 30-A | LKB1 |
| 9-C | WT1 |
| 60-E | ESR1 |
| 12-A | APC |
| 29-A | IGF2 |
| 8-D | MYOD1 |
| 36-A | N33 |
| 54-A | TPEF (=TMEFF2; =HPP1) |
| 18-E | CDKN2a |
| 15-D | CDH1 |
| 12-C | APC |

Healthy vs Carcinoma

| | |
|---|---|
| 50-D | CDH13 |
| 54-C | TPEF (=TMEFF2; =HPP1) |
| 50-C | CDH13 |
| 21-C | CSPG2 |
| 20-C | CD44 |
| 24-B | ERBB2 |
| 12-A | APC |
| 52-C | PTGS2 |
| 24-D | ERBB2 |
| 39-B | PGR |
| 25-B | GSTP1 |
| 49-A | CAV1 |
| 23-E | EGFR |
| 36-B | N33 |
| 29-C | IGF2 |
| 10-D | HLA-F |
| 54-B | TPEF (=TMEFF2; =HPP1) |
| 46-A | TP73 |

Healthy vs Adenoma

| | |
|---|---|
| 20-C | CD44 |
| 10-A | HLA-F |
| 43-C | TGFBR2 |
| 26-A | GTBP/MSH6 |
| 26-B | GTBP/MSH6 |
| 30-B | LKB1 |
| 20-A | CD44 |
| 36-C | N33 |
| 50-D | CDH13 |
| 46-A | TP73 |
| 39-D | PTEN |
| 36-B | N33 |
| 54-C | TPEF (=TMEFF2; =HPP1) |
| 25-B | GSTP1 |
| 23-A | EGFR |
| 40-A | RARB |
| 36-D | N33 |
| 49-A | CAV1 |
| 54-B | TPEF (=TMEFF2; =HPP1) |
| 18-E | CDKN2a |
| 36-A | N33 |
| 32-B | MLH1 |
| 12-C | APC |
| 21-C | CSPG2 |
| 15-E | CDH1 |
| 52-C | PTGS2 |
| 62-D | RASSF1 |
| 9-C | WT1 |
| 18-D | CDKN2a |
| 60-E | ESR1 |
| 29-D | IGF2 |
| 8-D | MYOD1 |
| 50-C | CDH13 |
| 4-C | EGR4 |
| 42-C | S100A2 |
| 22-D | DAPK1 |
| 31-E | MGMT |
| 24-D | ERBB2 |
| 56-A | CEA |
| 9-D | WT1 |
| 7-E | GPIb beta |
| 14-C | CALCA |
| 52-D | PTGS2 |
| 8-B | MYOD1 |
| 24-B | ERBB2 |
| 21-D | CSPG2 |
| 38-C | PGR |
| 58-A | PCNA |
| 34-D | MSH3 |
| 9-B | WT1 |
| 35-B | MYC |
| 27-C | HIC-1 |
| 52-B | PTGS2 |
| 23-E | EGFR |
| 30-A | LKB1 |
| 29-C | IGF2 |
| 39-C | PTEN |
| 13-D | BCL2 |
| 5-B | AR |
| 15-D | CDH1 |

Carcinoma vs Adenoma

| | |
|---|---|
| 18-B | CDKN2a |
| 7-E | GPIb beta |

Comparison of Healthy Colon Tissue with Non-Healthy Colon Tissue (Colon Adenoma and Colon Carcinoma):

FIG. 7 shows the differentiation according to the present invention, of healthy tissue from non-healthy tissue, where the non-healthy specimens are obtained from either colon adenoma or colon carcinoma tissue. The evaluation is carried out using informative CpG positions from 27 different genes as identified by the novel methods herein. Particular genes are further described in TABLE 4 above. The vertical ‘tick’ marks above and below the Figure demarcate the separation between tissue classes (i.e., between healthy and non-healthy).

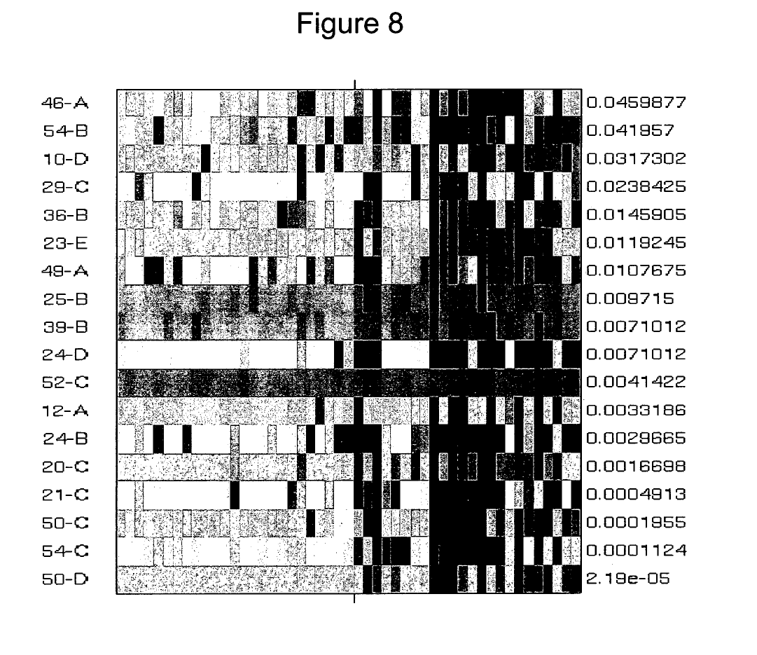

Healthy Colon Tissue Compared to Colon Carcinoma Tissue (FIG. 8):

FIG. 8 shows the differentiation of healthy tissue from carcinoma tissue using informative CpG positions from 15 genes, according to the present invention. The genes are further described in TABLE 4 above. The vertical ‘tick’ marks above and below the Figure demarcate the separation between tissue classes (i.e., between healthy and colon carcinoma).

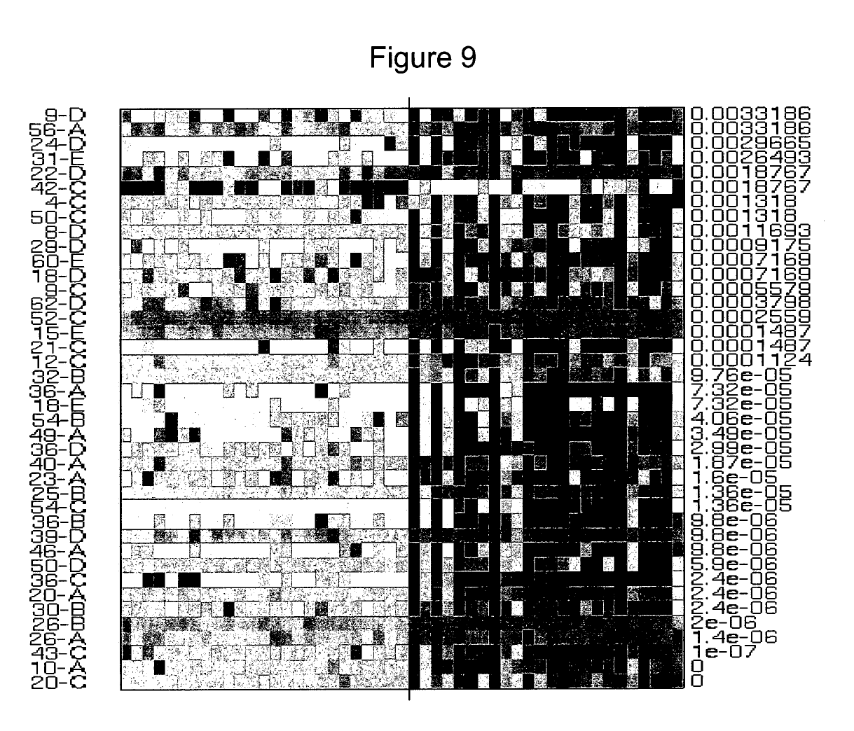

Healthy Colon Tissue Compared to Colon Adenoma Tissue (FIG. 9):

FIG. 9 shows the differentiation of healthy tissue from adenoma tissue using informative CpG positions from 40 genes. Informative genes are further described in Table 4. The vertical ‘tick’ marks above and below the Figure demarcate the separation between tissue classes (i.e., between healthy and colon adenoma).

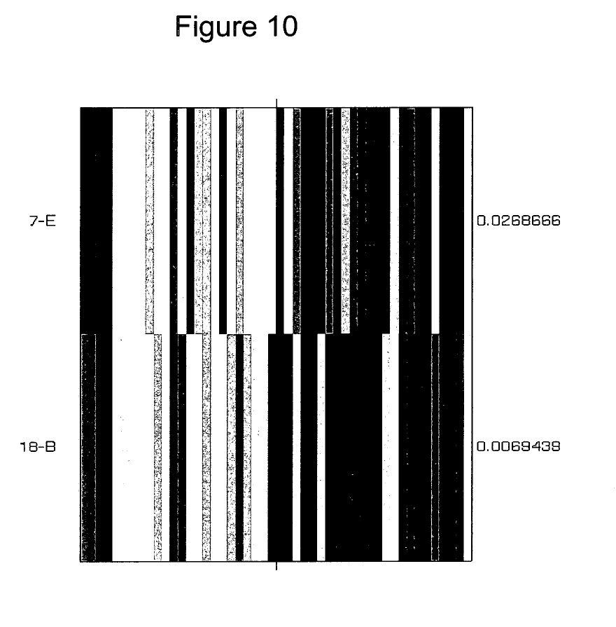

Colon Carcinoma Tissue Compared to Colon Adenoma Tissue (FIG. 10):

FIG. 10 shows the differentiation of carcinoma tissue from adenoma tissue using informative CpG positions from 2 genes. Informative genes are further described in Table 4. The vertical ‘tick’ marks above and below the Figure demarcate the separation between tissue classes (i.e., between colon carcinoma and colon adnenoma).

Step 5—Assay development and validation.

In this step of the method, two methodologies, namely MSP and MethylHeavy™, were evaluated as to their suitability for use as diagnostic platforms and to further validate the suitability of specific gene associated CpG positions as diagnostic markers for the analysis of colon cancer.

Both methodologies are used for the analysis of bisulphite-treated DNA, and both methods indicate the presence or absence of methylation-dependant sequences in the treated sequence during the post-bisulfite treatment amplification steps of the method. In both cases, said amplification is carried out by means of a polymerase chain reaction.

In the MSP technique, the use of methylation status-specific primers for the amplification of bisulphate-treated DNA allows the differentiation between methylated and unmethylated nucleic acids. MSP primer pairs contain at least one primer which hybridizes to a bisulphate-treated CpG dinucleotide. Therefore, the sequence of said primers comprises at least one CpG, TpG or CpA dinucleotide. MSP primers specific for non methylated DNA contain a ‘T’ at the 3′-position of the C position in the CpG. More preferably, said primers cover multiple CpG positions and thereby are most useful for the analysis of co-methylated regions. The methylation specific primers both prime the amplification reaction and contribute to the sensitivity of the reaction (see FIG. 4).

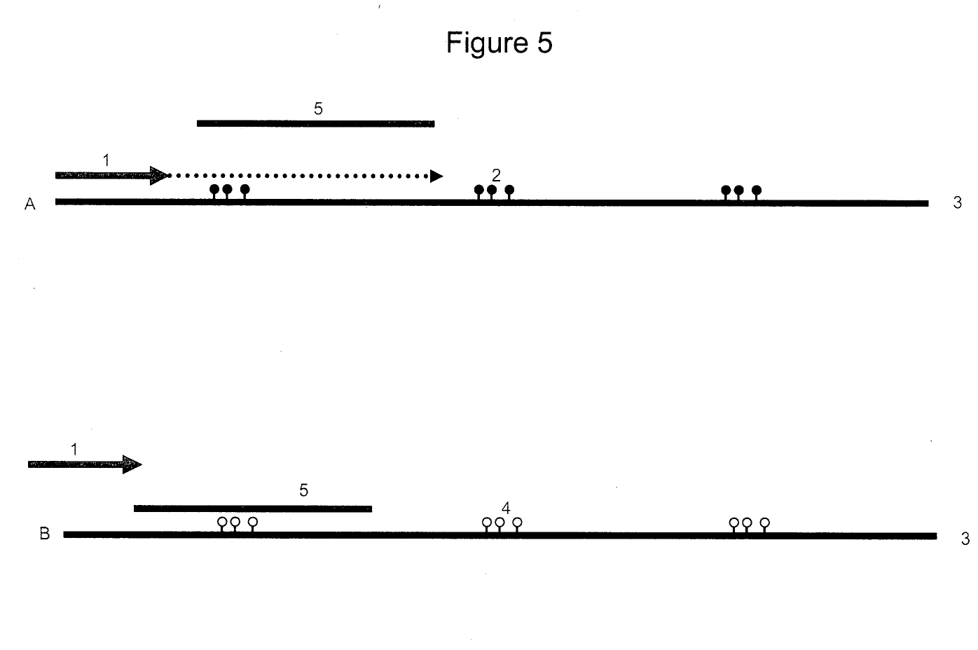

In the MethylHeavy™ technique, polymerase amplification is primed using methylation unspecific primers (i.e., the primers are designed to anneal to a sequence not containing any CpG or TpG dinucleotides), therefore the primers do not contribute to the methylation sensitivity of the assay. The methylation status of the bisulphite-treated CpG dinucleotides is determined by means of oligonucleotide blocking probes that are not displaced by the action of the polymerase, and thus block amplification of the sequence (see FIG. 5).

FIG. 5 shows polymerase-mediated amplification analysis of bisulfite-treated DNA (“3”) corresponding to a CpG-rich genomic sequence by means of the MethylHeavy™ technique. Amplification of the treated DNA (“3”) is precluded if the blocking oligonucleotide (“5”) anneals to the treated DNA as shown for the example case “B.” The arrows (“1”) represent primers, and dark circular marker positions (“2”) on the bisulfite-treated nucleic acid strand (“3”) represent methylated bisulfite-converted CpG positions, whereas white circular marker positions (“4”) represent unmethylated bisulfite-converted positions. The blocking (blocker) oligonucleotides are represented by dark bars (“5”). In the example case “A,” all subject genomic CpG positions were co-methylated, and both forward and reverse primers anneal to provide for unimpeded amplification of the corresponding treated nucleic acid (“3”). In the second example case “B,” none of the subject genomic CpG positions were methylated, both forward and reverse primers anneal to the treated DNA sequence (“3”) but are unable to amplify the sequence, because the synthesis of the complementary strand is blocked by the blocking oligonucleotide (“5”) that anneals to a complementary position comprising unmethylated CpG sequences in the subject genomic DNA.

In the following example, methylation patterns within the gene Calcitonin were analysed by means of the MethylLight™ and combined MethylLight™-HeavyMethyl™ assays.

In the first part of the example, a real-time PCR was carried out upon bisulphate-treated DNA and fluorescent labeled probes in a real-time PCR assay covering CpG positions of interest (a variant of the Taqman™ assay known as the ‘MethyLight™ assay).

In the second part of the example, methylation status of the same region was analysed by bisulphate-treatment, followed by analysis of the treated nucleic acids using a MethylLight™ assay combined with the methylation specific blocking probes covering CpG positions (HeavyMethyl™ assay).

Analysis of methylation of the gene Calcitonin within colon cancer using a MethyLight Assay. DNA was extracted from 34 colon adenocarcinoma samples and 42 colon normal adjacent tissues using a Qiagen extraction kit. The DNA from each sample was treated using a bisulfite solution (e.g., hydrogen sulfite, disulfite) according to the agarose bead method (Olek et al., 1996, supra). The treatment is such that all non methylated cytosines within the sample are converted to thymidine, whereas 5-methylated cytosines within the sample remain unmodified.

The methylation status was determined with a MethyLight™ assay designed for the CpG island of interest and a control fragment from the beta-actin gene (Eads et al., 2001 supra). The CpG island assay covers CpG sites in both the primers and the taqman style probe, while the control gene does not. The control gene is used as a measure of total DNA concentration, and the CpG island assay determines the methylation levels at that site. Primers and probe for the CpG island assay were as follows:

| | | |
|---|---|---|
| Primer: | AGGTTATCGTCGTGCGAGTGT; | (SEQ ID NO:7) |
| Primer: | TCACTCAAACGTATCCCAAACCTA; and | (SEQ ID NO:8) |
| Probe: | CGAATCTCTCGAACGATCGCATCCA. | (SEQ ID NO:9) |

Primers and probe for the beta-actin control assay were as follows:

| | |
|---|---|
| Primer: | |
| TGGTGATGGAGGAGGTTTAGTAAGT; | (SEQ ID NO:10) |
| Primer: | |
| AACCAATAAAACCTACTCCTCCCTTAA; and | (SEQ ID NO:11) |
| Probe: | |
| ACCACCACCCAACACACAATAACAAACACA. | (SEQ ID NO:12) |

The reactions were run in triplicate on each DNA sample with the following assay conditions:

Reaction solution: 900 nM primers; 300 nM probe; 3.5 mM magnesium chloride; 1 unit of taq polymerase; 200 μM dNTPs; and 7 μL of DNA, all in a final reaction volume was 20 μL.

Cycling conditions: 95° C. for 10 minutes, 95° C. for 15 seconds, 67° C. for 1 minute (3 cycles); 95° C. for 15 seconds, 64° C. for 1 minute (3 cycles); 95° C. for 15 seconds, 62° C. for 1 minute (3 cycles); and 95° C. for 15 seconds, 60° C. for 1 minute (40 cycles).

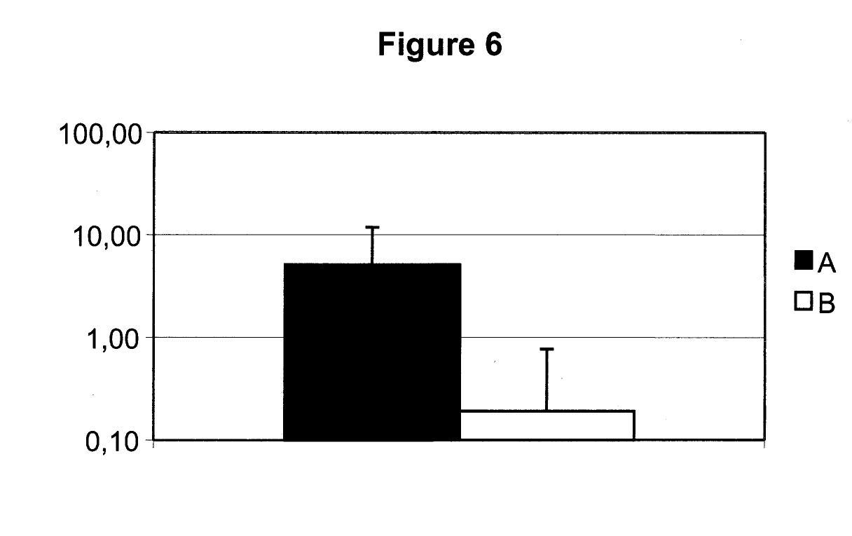

The data was analyzed using a PMR calculation previously described in the literature (Eads et al., 200, supra). The mean PMR for normal samples was 0.19 with a standard deviation of 0.79. None of the normal samples was greater than 2 standard deviations about the normal mean, while 18 of 34 tumor samples reached this level of methylation. The overall difference in methylation levels between tumor and normal samples is significant in a t-test (p=0.002) (see FIG. 6)

Analysis of methylation of the gene Calcitonin within colon cancer using a HeavyMethyl™ MethyLight™ Assay. The same DNA samples were also used to analyze methylation of the CpG island with a HeavyMethyl™ MethyLight™ (or HM MethyLight™) assay, also referred to as the HeavyMethyl™ assay. The methylation status was determined with a HM MethyLight™ assay designed for the CpG island of interest and the same beta-actin control gene assay described above. The CpG island assay covers CpG sites in both the blockers and the Taqman™ style probe, while the control gene does not. Primers and probes for the CpG island assay were as follows:

| | |
|---|---|
| Primer: | |
| GGATGTGAGAGTTGTTGAGGTTA; | (SEQ ID NO:13) |
| Primer: | |
| ACACACCCAAACCCATTACTATCT; and | (SEQ ID NO:14) |
| Probe: | |
| ACCTCCGAATCTCTCGAACGATCGC; | (SEQ ID NO:15) |
| Blocker: | |
| TGTTGAGGTTATGTGTAATTGGGTGTGA. | (SEQ ID:NO 16) |

The reactions were each run in triplicate on each DNA sample with the following reaction conditions:

Reaction solution: 300 nM primers; 450 nM probe; 3.5 mM magnesium chloride; 2 units of taq polymerase; 400 μM dNTPs; and 7 μL of DNA; all in a final reaction volume of 20 μL.

Cycling conditions: 95° C. for 10 minutes, 95° C. for 15 seconds, 67° C. for 1 minute (3 cycles); 95° C. for 15 seconds, 64° C. for 1 minute (3 cycles); 95° C. for 15 seconds, 62° C. for 1 minute (3 cycles); and 95° C. for 15 seconds, 6° C. for 1 minute (40 cycles).

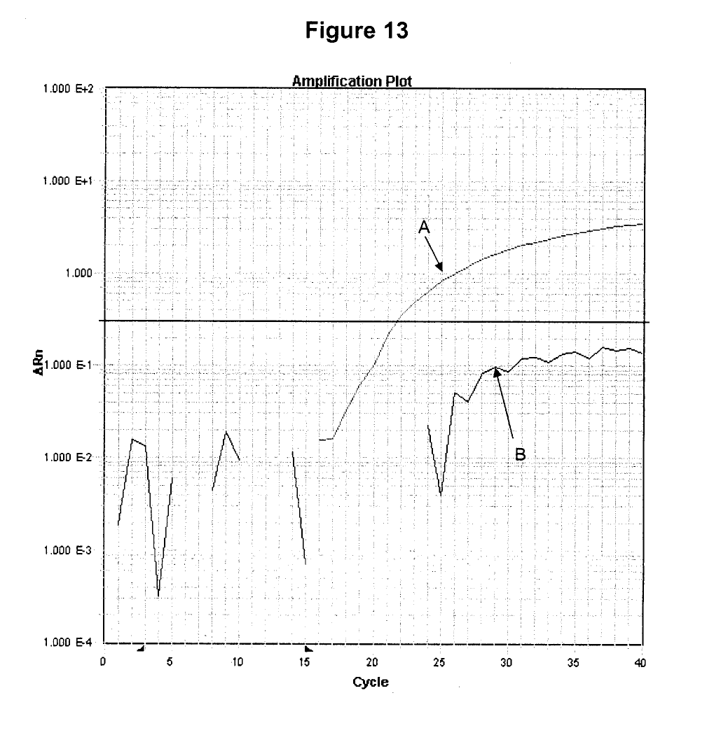

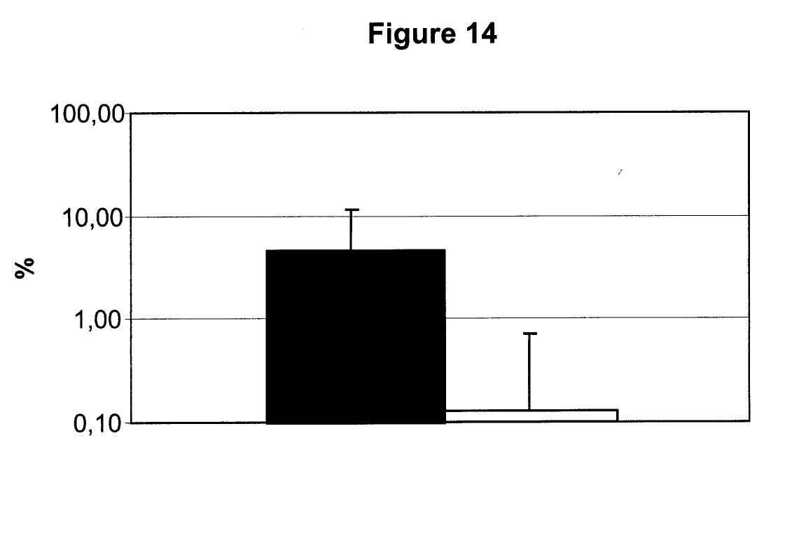

The mean PMR for normal samples was 0.13 with a standard deviation of 0.58. None of the normal samples was greater than 2 standard deviations about the normal mean, while 19 of 34 tumor samples reached this level of methylation. The overall difference in methylation levels between tumor and normal samples is significant in a t-test (p=0.0004) (see FIGS. 13 and 14).

Therefore, the two methodologies MSP and MethylHeavy™, were evaluated herein and shown to be suitable for use as applied diagnostic platforms, and represent further validation of the suitability of specific gene associated CpG positions as, inter alia, diagnostic markers for diagnostic, prognostic, and staging of cancer, including colon cancer.

1. A method for identification of a reliable diagnostic, prognostic or staging marker for phenotypic conditions characterized by altered DNA methylation, comprising:
a) obtaining a set of at least two biological samples in each case having genomic DNA, wherein the biological samples correspond to at least two sample classes that are distinguishable by at least one of a phenotypic or measurable parameter;
b) identifying, using a genome-wide assay or discovery technique suitable for comparing methylation status between or among corresponding CpG dinucleotide positions within the respective sample class genomic DNA samples, a plurality of primary differentially methylated CpG dinucletide sequence positions;
c) selecting at least one of the primary differentially methylated CpG dinucletide sequence positions, based on scoring thereof according to likely utility for discriminating between said at least two sample classes; and
d) confirming, as among a larger set of such biological samples, and using an assay suitable therefore, the class-distinguishing methylation status of at least one such selected primary differentially methylated CpG dinucleotide sequence position, whereby a reliable methylation marker for at least one of diagnosis, prognosis or staging is provided.

2. The method of claim 1, further comprising, prior to confirming in d), identifying within a context DNA region surrounding or including one of the primary differentially methylated CpG dincleotide positions, and using an assay or database suitable therefore, at least one secondary differentially methylated CpG dinucleotide sequence, and wherein confirming the class-distinguishing methylation status in d) further comprises confirming the class-distinguishing methylation status of the at least one secondary differentially methylated CpG dinucleotide sequence position.

3. The method of claim 2, wherein the classes are distinguished, based on the secondary differentially methylated CpG dinucleotide sequence position alone, or in combination with other differentially methylated CpG dinucleotide sequence CpG positions.

4. The method of any one of claims 1 or 2, wherein confirming in d) comprises use of at least one of a suitable medium- or a high-throughput assay.

5. The method of claim 1, wherein the phenotypic parameter is selected from the group consisting of cell proliferative disorders; metabolic malfunctions or disorders; immune malfunctions, damage or disorders; CNS malfunctions, damage or disease; symptoms of aggression or behavioural disturbances; clinical, psychological and social consequences of brain damage; psychotic disturbances and personality disorders; dementia or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, the muscles, the connective tissue or the bones; endocrine and metabolic malfunction, damage or disease; headaches or sexual malfunction, treatment or pharmacological response; age; life style; disease history; signaling chains; protein synthesis; behavior; drug abuse; patient history; cellular parameters; histological parameters, physiological parameters; anatomical parameters; pathological parameters; treatment history, gene expression, and combinations thereof.

6. The method of claim 1, wherein the biological sample classes are distinguishable by two or more phenotypic parameters.

7. The method of claim 1, wherein at least one of identifying in b) or confirming in d) is by use of phenotypically matched sets or pools of biological samples of each class.

8. The method of claim 1, wherein the biological sample source of the genomic DNA is selected from the group consisting of cells, cellular components comprising genomic DNA, cell lines, tissue biopsies, bodily fluids, blood, serum, sputum, stool, urine, ejaculate, cerebrospinal fluid, paraffin-embedded tissue, histological object slides, and combinations thereof.

9. The method of claim 1, wherein identifying in b) comprises use a methylation-sensitive restriction enzyme based technique.

10. The method of claim 9, wherein the methylation-sensitive restriction enzyme based technique is selected from the group consisting of methylated CpG island amplification, arbitrarily-primed polymerase chain reaction, restriction landmark genomic scanning, differential methylation hybridization, Not I restriction-based differential methylation hybridization, and combinations thereof.

11. The method of claim 1, wherein identifying in b) comprises analysis of at least 50 different CpG positions.

12. The method of claim 1, wherein identifying in b) comprises analysis of a plurality of CpG positions corresponding in genomic position to at least 20 genes, or to their respective promoters, introns, first exons, second exons, or enhancers.

13. The method of claims 1, further comprising, in at least one of b), c) or d), assessing the primary differentially methylated CpG dinucleotide sequence position according to at least one additional parameter, wherein a subset of the primary differentially methylated CpG dinucleotide sequence positions are selected for progression through subsequent steps.

14. The method of claim 13, wherein the at least one additional parameter is selected from the group consisting of: confirmation of the differentially methylated CpG position using multiple techniques; tissue specificity of the differentially methylated CpG position; sequence context of the differentially methylated CpG position; presence of a gene associated with the location of the differentially methylated CpG position; and combinations thereof.

15. The method of claim 2, wherein identifying within a context DNA region comprises use of bisulfite treatment of the DNA, and sequencing of the treated DNA.

16. The method of claim 15, wherein the sequencing comprises one or more techniques selected from the group consisting of a Sanger-based method, a Maxam-Gilbert-based method, sequencing by hybridization (SBH), and combinations thereof.

17. The method of claims 1, wherein confirming in d) comprises use of a technique selected from the group consisting of oligonucleotide hybridization analysis, MS-SnuPE, and combinations thereof.

18. The method of claim 1, wherein confirming in d) comprises:
a) obtaining a biological sample containing genomic DNA;
b) extracting the genomic DNA;
c) treating the genomic DNA to convert cytosine bases that are unmethylated at the C5-position to uracil or to another base which is detectably dissimilar to cytosine in terms of hybridization properties;
d) amplifying fragments of the treated genomic DNA using sets of primer oligonucleotides and a polymerase; and
e) identifying the methylation status of one or more CpG dinucleotide positions.

19. The method of claim 18, comprising amplification of at least 10 different DNA fragments, having, in each case, a length of about 100 to about 2000 nucleotides.

20. The method of claim 18, wherein amplification comprises amplification of several DNA segments in one reaction vessel.

21. The method of claim 18, wherein the polymerase is a heat-resistant DNA polymerase.

22. The method of claim 18, wherein amplification comprises use of a polymerase chain reaction (PCR).

23. The method of claim 18, comprising labeling of amplificates using a label selected from the group consisting of: fluorescence labels; radionuclides or radiolabels; mass labels; detachable molecule fragments having a characteristic mass detectable in a mass spectrometer; detachable molecule fragments having a single-positive or single-negative charge and detectable in a mass spectrometer; and combinations thereof.

24. The method of claim 18, comprising detection of amplificates, or fragments thereof in a mass spectrometer.

25. The method of claim 24, wherein detection in the mass spectrometer comprises use of matrix assisted laser desorption/ionization mass spectrometry (MALDI), electron spray mass spectrometry (ESI), or combinations thereof.

26. The method of claim 18, wherein identifying the methylation status of one or more CpG dinucleotide positions in e) comprises hybridization of at least one oligonucleotide.

27. The method of claim 18, wherein identifying the methylation status of one or more CpG dinucleotide positions in e) comprises hybridization of an oligonucleotide and extension of the hybridized oligonucleotide by means of at least one nucleotide base.

28. The method of claim 26, wherein at least one oligonucleotide is immobilized on a solid phase.

29. The method of clam 28, wherein the solid phase comprises a material selected from the group consisting of silicon, glass, polystyrene, aluminum, steel, iron, copper, nickel, silver, gold, and combinations thereof.

30. The method of claim 1, wherein confirming in d) comprises training a machine learning algorithm to distinguish between the two classes of phenotypes.

31. The method of claim 1, further comprising, in a step (e), development of an applied assay for diagnostic use of the identified markers.

32. The method of claim 31, wherein the applied assay comprises an assay selected from the group consisting of MSP, MethyLight™, HeavyMethyl™, MS-SnuPE, and combinations thereof.

33. The method of claim 31, wherein the applied assay comprises:
i) treating of the DNA to convert unmethylated cytosine bases to uracil, or to another base which is detectably dissimilar to cytosine in terms of hybridization properties, wherein 5-methylcytosine bases remain unconverted;
ii) amplifying of one or more nucleic acid fragments comprising one or more CpG positions identified in d) using at least 2 primer oligonucleotides;
iii) detecting of the amplificate nucleic acids;
iv) determining of the methylation state of said CpG positions; and
v) correlating the methylation state to one or more of the phenotypic or measurable parameters defined in a).

34. The method of claim 33, wherein treating in i) comprises use of a bisulfite reagent.

35. The method of claim 34, wherein treating in i) is subsequent to embedding the DNA in agarose.

36. The method of claim 34, where treating in i) comprises treating in the presence of at least one of a DNA denaturing reagent or a radical trap reagent.

37. The method of claim 33, wherein amplifying in ii) comprises at least one of preferential amplification of CpG positions that were methylated prior to treatment relative to amplification of positions that were unmethylated prior to treatment, or preferential amplification of positions that were unmethylated prior to treatment relative to amplification of positions that were methylated prior to treatment.

38. The method of claim 37, wherein amplifying comprises amplification of at least 6 different fragments.

39. The method of claim 33, further comprising, subsequent to treating in i), use of at least one oligonucleotide or peptide nucleic acid (PNA) oligomer which hybridizes to said one or more CpG positions confirmed in d), wherein said oligonucleotide preferentially hybridizes to at least one of positions that were methylated prior to treatment, or to positions that were unmethylated prior to treatment.

40. The method of claim 33, wherein at least one of the primers comprises a characteristic selected from the group consisting of: being at least 18 nucleotides in length; having a 5′-CpG-3′ dinucleotide; having a 5′-TpG-3′ dinucleotide; having a 5′-CpA-3′-dinucleotide; having a 5′-CpG-3′ dinucleotide in the middle one third of the primer; having a 5′-TpG-3′ dinucleotide in the middle one third of the primer; having a 5′-CpA-3′-dinucleotide in the middle one third of the primer; and combinations thereof.

41. The method of claim 39, wherein the at least one of the oligonucleotides or PNA oligomers comprise a characteristic selected from the group consisting of: being at least 18 nucleotides in length; having a 5′-CpG-3′ dinucleotide; having a 5′-TpG-3′ dinucleotide; having a 5′-CpA-3′-dinucleotide; having a 5′-CpG-3′ dinucleotide in the middle one third of the oligonucleotide or PNA oligomer; having a 5′-TpG-3′ dinucleotide in the middle one third of the oligonucleotide or PNA oligomer; having a 5′-CpA-3′-dinucleotide in the middle one third of the oligonucleotide or PNA oligomer; and combinations thereof.

42. The method of claim 39, wherein the binding site of the oligonucleotide or PNA oligomer is identical to, or overlaps with that of the primer and thereby hinders hybridization of the primer to its binding site.

43. The method of claim 42, wherein amplification of the background DNA is hindered.

44. The method of claim 43, wherein amplification of DNA that was unmethylated prior to treatment of the unmethylated cytosine-containing DNA is hindered.

45. The method of claim 42, wherein the binding sites of at least two of the oligonucleotides or PNA oligomers are identical to, or overlap with those of at least two of the primers, and thereby hinder hybridization of the primers to their binding site.

46. The method of claim 45, wherein hybridization of at least one of the oligonucleotides or peptide nucleic acid oligomers hinders hybridization of a forward primer, and the hybridization of at least one of the oligonucleotides or peptide nucleic acid oligomers hinders the hybridization of a reverse primer that binds to the elongation product of said forward primer

47. The method of claim 42, wherein said oligonucleotide or peptide nucleic acid oligomer hybridizes between the binding sites of the forward and reverse primers.

48. The method of claim 42, wherein said oligonucleotide or PNA oligomer preferentially hybridizes to either positions that were methylated prior to treatment, or preferentially hybridizes to positions that were unmethylated prior to treatment.

49. The method of claim 42, wherein the oligonucleotide concentration exceeds that of the primer oligonucleotides by at least 5-fold.

50. The method of claim 42, wherein the polymerase used has no 5′-3′ exonuclease activity.

51. The method of claim 42, wherein the oligonucleotides or PNA oligomers are modified at the 5′ end to preclude degredation by a polymerase with 5′-3′ exonuclease activity.

52. The method of claim 42, wherein the probe oligonucleotides or peptide nucleic acid oligomers lack a free 3′-hydroxyl group.

53. The method of claim 42, wherein detection of the amplificate nucleic acids in iii) comprises use of at least one reporter oligonucleotide that hybridizes to a 5′-CpG-3′ dinucleotide, or to a 5′-TpG-3′ dinucleotide, or to a 5′-CpA-3′ dinucleotide.

54. The method of claim 42, wherein amplification in ii) comprises use of at least one blocking oligonucleotide or PNA oligomer that hybridizes to a 5′-CpG-3′ dinucleotide, or to a 5′-TpG-3′ dinucleotide, or to a 5′-CpA-3′ dinucleotide, and thereby hinders amplification of at least one nucleic acid sequence that was either methylated prior to treating in i), or unmethylated prior to treating in step i), and wherein detecting in iii) comprises at least one reporter oligonucleotide, which hybridizes to a 5′-CpG-3′ dinucleotide, or to a 5′-TpG-3′ dinucleotide, or to a 5′-CpA-3′ dinucleotide.

55. The method of claim 53, further comprising the use of a fluorescent labeled oligomer that hybridizes directly adjacent to the reporter oligonucleotide, wherein said hybridization is detectable by fluorescence resonance energy transfer, and wherein the detection is by either an increase or a decrease in fluorescence.

56. The method of claim 53, wherein the reporter oligonucleotides are fluorescently labeled, and wherein detection thereof is by either an increase or a decrease in fluorescence.

57. The method of any one of claims 55 or 56, wherein the methylation state of one or more CpG positions of the DNA prior to treatment is determined based on an increase or decrease in fluorescence.

58. The method of claim 43, wherein the background DNA concentration is at about a 100-fold excess of the concentration of the DNA to be investigated, or is at about a 1,000-fold excess of the concentration of the DNA to be investigated.

59. The method of any one of claims 33, 42 or 53, comprising use of at least one of a TaqMan™ assay, or LightCycler™ assay.

60. The method of claim 33, wherein determining of the methylation state of the CpG positions in iv) comprises use of an MS-SnuPE reaction.

61. The method of claim 60, wherein the Ms-SnuPE primer is at least fifteen but no more than twenty five nucleotides in length.

62. The method of claim 33, wherein correlating the methylation state to one or more of the phenotypic parameters in v) comprises the use of a machine learning algorithm.

63. The method of claim 62, wherein the machine learning algorithm comprises a linear classifier.

64. The method of claim 62, wherein the machine learning algorithm is selected from the group consisting of support vector machines (SVM), perceptrons, Bayes Point Machines, and combinations thereof.

65. A diagnostic, prognostic or staging kit, useful to practice the method according to claim 32, and comprising at least one primer having a characteristic selected from the group consisting of: being at least 18 nucleotides in length; having a 5′-CpG-3′ dinucleotide; having a 5′-TpG-3′ dinucleotide; having a 5′-CpA-3′-dinucleotide; having a 5′-CpG-3′ dinucleotide in the middle one third of the primer; having a 5′-TpG-3′ dinucleotide in the middle one third of the primer; having a 5′-CpA-3′-dinucleotide in the middle one third of the primer; and combinations thereof.

66. A diagnostic, prognostic or staging kit, useful to practice the method according to claim 33, and comprising at least one oligonucleotide or PNA oligomer having a characteristic selected from the group consisting of: being at least 18 nucleotides in length; having a 5′-CpG-3′ dinucleotide; having a 5′-TpG-3′ dinucleotide; having a 5′-CpA-3′-dinucleotide; having a 5′-CpG-3′ dinucleotide in the middle one third of the oligonucleotide or PNA oligomer; having a 5′-TpG-3′ dinucleotide in the middle one third of the oligonucleotide or PNA oligomer; having a 5′-CpA-3′-dinucleotide in the middle one third of the oligonucleotide or PNA oligomer; and combinations thereof.

67. A diagnostic, prognostic or staging method, comprising: use of the method according to claim 1, or a kit according to claim 66, for characterization, classification, differentiation, grading, staging, diagnosis, or prognosis of a condition selected from the group consisting of unwanted side effects of medicaments, cell proliferative disorders or predisposition to cell proliferative disorders; metabolic malfunctions or disorders; immune malfunctions, damage or disorders; CNS malfunctions, damage or disease; symptoms of aggression or behavioural disturbances; clinical, psychological and social consequences of brain damage; psychotic disturbances and personality disorders; dementia or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, the muscles, the connective tissue or the bones; endocrine and metabolic malfunction, damage or disease; headaches or sexual malfunction, treatment or pharmacological response; age; life style; disease history; signaling chains; protein synthesis; behavior; drug abuse; patient history; cellular parameters; histological parameters, physiological parameters; anatomical parameters; pathological parameters; treatment history, gene expression, and combinations thereof.

68. The method of claim 67, wherein the diagnosis or prognosis is selected from the group consisting of leukaemia, head and neck cancer, Hodgkin's disease, gastric cancer, prostate cancer, renal cancer, bladder cancer, breast cancer, Burkitt's lymphoma, Wilms tumor, Prader-Willi/Angelman syndrome, ICF syndrome, dermatofibroma, hypertension, pediatric neurobiological diseases, autism, ulcerative colitis, fragile-X syndrome, Huntington's disease, and combinations thereof.

69. A method for the treatment of a disease or medical condition, comprising:
a) providing at least one diagnosis or prognosis of a condition according to the method of claim 67; and
b) specifying a suitable treatment therefore.


SEQUENCE LISTING (40 sequences)

SEQ ID NO: 1 - DNA, 24 bp, artificial sequence
  RMXA24 adapter-primer
agcactctccagcctctcaccgac

SEQ ID NO: 2 - DNA, 12 bp, artificial sequence
  RMXA12 adapter-primer
ccgggtcggtga

SEQ ID NO: 3 - DNA, 24 bp, artificial sequence
  JXMA24 adapter-primer
accgacgtcgactatccatgaacc

SEQ ID NO: 4 - DNA, 12 bp, artificial sequence
  JXMA12 adapter-primer
ccggggttcatg

SEQ ID NO: 5 - DNA, 24 bp, artificial sequence
  NXMA24 adapter-primer oligonucleotide
aggcaactgtgctatccgagtgac

SEQ ID NO: 6 - DNA, 12 bp, artificial sequence
  NXMA12 adapter-primer oligonucleotide
ccgggtcactcg

SEQ ID NO: 7 - DNA, 21 bp, artificial sequence
  calcitonin gene-specific forward primer
aggttatcgtcgtgcgagtgt

SEQ ID NO: 8 - DNA, 24 bp, artificial sequence
  calcitonin gene-specific reverse primer
tcactcaaacgtatcccaaaccta

SEQ ID NO: 9 - DNA, 25 bp, artificial sequence
  calcitonin gene-specific probe
cgaatctctcgaacgatcgcatcca

SEQ ID NO: 10 - DNA, 25 bp, artificial sequence
  beta-actin specific forward primer
tggtgatggaggaggtttagtaagt

SEQ ID NO: 11 - DNA, 27 bp, artificial sequence
  beta-actin specific reverse primer
aaccaataaaacctactcctcccttaa

SEQ ID NO: 12 - DNA, 30 bp, artificial sequence
  beta-actin specific probe
accaccacccaacacacaataacaaacaca

SEQ ID NO: 13 - DNA, 23 bp, artificial sequence
  calcitonin gene-specific forward primer
ggatgtgagagttgttgaggtta

SEQ ID NO: 14 - DNA, 24 bp, artificial sequence
  calcitonin gene-specific reverse primer
acacacccaaacccattactatct

SEQ ID NO: 15 - DNA, 25 bp, artificial sequence
  calcitonin gene-specific probe
acctccgaatctctcgaacgatcgc

SEQ ID NO: 16 - DNA, 28 bp, artificial sequence
  calcitonin gene-specific blocker oligonucleotide
tgttgaggttatgtgtaattgggtgtga

SEQ ID NO: 17 - DNA, 10 bp, artificial sequence
  AP-PCR Primer CG1
gggccgcggc

SEQ ID NO: 18 - DNA, 10 bp, artificial sequence
  AP-PCR Primer CG2
ccccgcgggg

SEQ ID NO: 19 - DNA, 10 bp, artificial sequence
  AP-PCR Primer CG3
cgcgggggcg

SEQ ID NO: 20 - DNA, 10 bp, artificial sequence
  AP-PCR Primer CG4
gcgcgccgcg

SEQ ID NO: 21 - DNA, 10 bp, artificial sequence
  AP-PCR Primer CG5
gcggggcggc

SEQ ID NO: 22 - DNA, 10 bp, artificial sequence
  AP-PCR Primer G1
gcgccgacgt

SEQ ID NO: 23 - DNA, 10 bp, artificial sequence
  AP-PCR Primer G2
cgggacgcga

SEQ ID NO: 24 - DNA, 10 bp, artificial sequence
  AP-PCR Primer G3
ccgcgatcgc

SEQ ID NO: 25 - DNA, 10 bp, artificial sequence
  AP-PCR Primer G4
tggccgccga

SEQ ID NO: 26 - DNA, 10 bp, artificial sequence
  AP-PCR Primer G5
tgcgacgccg

SEQ ID NO: 27 - DNA, 10 bp, artificial sequence
  AP-PCR Primer G6
atcccgcccg

SEQ ID NO: 28 - DNA, 10 bp, artificial sequence
  AP-PCR Primer G7
gcgcatgcgg

SEQ ID NO: 29 - DNA, 10 bp, artificial sequence
  AP-PCR Primer G8
gcgacgtgcg

SEQ ID NO: 30 - DNA, 10 bp, artificial sequence
  AP-PCR Primer G9
gccgcgngng

SEQ ID NO: 31 - DNA, 10 bp, artificial sequence
  AP-PCR Primer G10
gcccgcgnng

SEQ ID NO: 32 - DNA, 10 bp, artificial sequence
  AP-PCR Primer APBS1
agcggccgcg

SEQ ID NO: 33 - DNA, 10 bp, artificial sequence
  AP-PCR Primer APBS5
ctcccacgcg

SEQ ID NO: 34 - DNA, 10 bp, artificial sequence
  AP-PCR Primer APBS7
gaggtgcgcg

SEQ ID NO: 35 - DNA, 10 bp, artificial sequence
  AP-PCR Primer APBS10
aggggacgcg

SEQ ID NO: 36 - DNA, 10 bp, artificial sequence
  AP-PCR Primer APBS11
gagaggcgcg

SEQ ID NO: 37 - DNA, 10 bp, artificial sequence
  AP-PCR Primer APBS12
gcccccgcga

SEQ ID NO: 38 - DNA, 10 bp, artificial sequence
  AP-PCR Primer APBS13
cggggcgcga

SEQ ID NO: 39 - DNA, 10 bp, artificial sequence
  AP-PCR Primer APBS17
ggggacgcga

SEQ ID NO: 40 - DNA, 10 bp, artificial sequence
  AP-PCR Primer APBS18
accccacccg